(19) United States
(12) Patent Application Publication
(10) Pub. No.: US 2025/0188626 A1
(43) Pub. Date: Jun. 12, 2025

(54) CARBON DIOXIDE CAPTURE AND UTILIZATION AS A CLEAN FEEDSTOCK

(71) Applicant: Michigan Technological University, Houghton, MI (US)

(72) Inventors: Surendra Komar KAWATRA, Fort Meyers, FL (US); Sriram VALLURI, Grayslake, IL (US)

(21) Appl. No.: 18/842,106

(22) PCT Filed: Apr. 26, 2023
(86) PCT No.: PCT/US2023/066241

§ 371 Date: Aug. 28, 2024

Related U.S. Application Data
(60) Provisional application No. 63/334,909, filed on Apr. 26, 2022.

(51) Int. Cl. C25B 3/26; B01D 53/62; B01D 53/80; B01D 53/96; B01D 61/44; C02F 1/66; C02F 103/16; C25B 3/07; C25B 9/17; C25B 11/02; C25B 11/075

(52) U.S. Cl. CPC C25B 3/26; B01D 53/62; B01D 53/80; B01D 53/96; B01D 53/965; B01D 61/445; C25B 3/07; C25B 9/17; C25B 11/02; C25B 11/075; B01D 2251/304; B01D 2251/606; B01D 2252/60; B01D 2257/504; B01D 2258/0283; C02F 1/66; C02F 2103/16

(57) ABSTRACT

Captured carbon dioxide, preferably carbon dioxide captured from emissions such as carbon dioxide from flue gas emissions, can be provided in the form of purified carbon dioxide after undergoing one or more purification processes, such that the purified carbon dioxide is capable of being utilized in the formation of one or more other economically viable chemicals, such as an oxalate salt and/or oxalic acid via an electrochemical reduction process. The electrochemical reduction process utilizing a modified cathode having a metal coating for the efficient and high yield of oxalate salt and/or oxalic acid. The economically viable chemicals derived from captured carbon dioxide capable of being utilized in other applications including the treatment of waste-products, such as in the neutralization of red mud and/or the extraction of rare earth minerals from neutralized red mud.

### CLAIM FOR PRIORITY

This application is as National Phase entry of PCT Application No. PCT/US2023/066241 filed Apr. 26, 2023, which claims the benefit of priority of U.S. Provisional Patent Application No. 63/334,909 filed Apr. 26, 2022, the subject matter of which is incorporated by reference in their entirety.

### TECHNICAL FIELD

The present invention relates generally to the utilization of captured CO₂, particularly captured CO₂ from an emission source, wherein the captured CO₂ has undergone one or more other processes to provide cleaned, captured CO₂ as a feedstock that can be utilized in one or more other processes, the one or more other processes including the generation of other chemicals, such as an oxalate acid and/or oxalic acid, and/or the neutralization of red mud and/or extraction of one or more rare earth minerals from red mud.

### BACKGROUND

As the world moves towards clean energy initiative, carbon capture and utilization technologies are key to achieving net zero emissions. As carbon dioxide (CO₂) emissions rise, CO₂ capture and utilization technologies have been deemed necessary to reduce pollution and mitigate the related climate effects. The goal of CO₂ capture technology is to provide a method of isolating CO₂ and reducing its emissions to the environment. The ideal long-term goal of such emissions reduction is to reach net negative emissions, where human activities balance out or are result in the net removal of CO₂ from the atmosphere. CO₂ utilization seeks to make this an economic and viable prospect by putting the CO₂ to work in stable and valuable tasks. Several avenues of CO₂ utilization are under investigation, including the transformation of CO₂ into valuable chemicals, high energy fuels, or directly into a plethora of working conditions.

Direct utilization uses the CO₂ as-is, without chemical conversion to other products. Widespread direct uses of CO₂ include use in food and beverages, fire extinguishers, concrete building materials, and CO₂ enhanced oil recovery. Indirect utilization uses the CO₂ as a feedstock in creating a more complex final product. Indirect utilization techniques primarily include the conversion of CO₂ to useful chemicals or fuels. The conversion of CO₂ to high energy density fuels is an attractive option to meeting the energy storage demands facing renewable energy.

The major challenge associated with utilizing CO₂ from waste streams is the cost of capturing it from those streams as opposed to acquiring CO₂ from natural sources. Large amounts of CO₂ can be obtained directly from natural gas reservoirs and industrial emissions, but in many cases the former has an economic advantage over the latter.

Flue gas emissions—the emitted material produced when fossil fuels such as coal, oil, natural gas, or wood are burned for heat or power—may contain pollutants, including CO₂, nitrogen oxides (NOₓ) and sulfur oxides (SOₓ). Capturing flue gasses from power plants is typically a multi-step process. This is usually done in three stages: (1) selective catalytic reduction (SCR) for the removal of NOx; (2) flue gas desulfurization (FGD) for the capture of SO₂ and (3) CO₂ capture.

Reagent regeneration is the most energy intensive step in post combustion CO₂ capture process. One approach to reagent regeneration is thermal regeneration as shown in FIG. 9, which comprises heating the resultant capture solution, such as sodium bicarbonate, to decompose the resultant capture solution to release CO₂ and regenerate the starting absorbent, such as sodium carbonate, which can then be recycled and reused for CO₂ capture from a flue gas. However, thermal regeneration can cost a massive amount of energy, which can be greater than 3.0 MJ/kg of CO₂ captured.

The utilization of captured CO₂ is another important consideration. Extensive research has been done on the chemistry of transforming CO₂ into more useful products. Methods are known to convert CO₂ into a wide variety of substances, including methanol, isobutanol, carbohydrates, methane, carbonates, urea, formic acid, oxalic acid, carbon monoxide, epoxides, formaldehyde, and so on. Several of these (carbohydrates, formaldehyde, isobutanol etc.) are primarily results from biological processes. The rest are results of strong reduction reactions or electrolytic reduction. Electrolytic reduction can be used to form methane, methanol, formic acid, oxalic acid and/or carbon monoxide from CO₂.

CO₂ reduces at the cathode in an electrolysis cell. These processes have the general form of generating the CO₂ anion radical (CO₂) and allowing it to react with itself or the electrolyte. Catalysts can be added to influence the formation of the anion radical or to suppress side reactions. The electrolyte, catalyst, voltage, electrode material and CO₂ content are all known to affect the reaction pathway. Table 1 shows the overall reactions and electrical energy (EE) requirements for electrochemical conversion of CO₂ to chemicals, as reported by Malik et al., Electrochemical reduction of CO₂ for synthesis of green fuel. Wiley Interdisciplinary Reviews: Energy and Environment 6(4), e2244 (2017) and Qiao et al., A review of catalysts for the electroreduction of CO₂ to produce low-carbon fuels. Chemical Society Reviews 43(2), 631-675 (2014).

TABLE 1
Energy cost for Producing Products from Carbon Dioxide.

| Product | Overall Reaction | EE (MJ/Kg) |
|---|---|---|
| Oxalate | 2CO₂ → C₂O₄²⁻ | 2.68 |
| Formic acid | 2CO₂ + 2H₂O → 2HCOOH + O₂ | 5.51 |
| Carbon monoxide | 2CO₂ + H₂O → 2CO + O₂ + H₂O | 9.19 |
| (Syn-gas) | | |
| Methanol | CO₂ + 2H₂O → 1.5O₂ + CH₃OH | 21.71 |
| Ethanol | 2CO₂ + 3H₂O → C₂H₅OH + 3O₂ | 28.70 |
| Propanol | 3CO₂ + 4H₂O → C₃H₇OH + 4.5O₂ | 29.53 |
| Methane | CO₂ + 2H₂O → 2O₂ + CH₄ | 51.15 |

Accordingly, there is a need in the industry to decrease reagent cost for post-combustion CO₂ capture. There is also a need in the industry to provide reagents that can be utilized in a slurry solution that has an acceptable absorption rate of CO₂ capture. There is further a need in the industry to provide a slurry solution that utilizes low energy for reagent regeneration, such that the reagent can be recycled and reused for acceptable absorption of CO₂ capture. There is even further a need in the industry to provide a slurry solution that does not result in undesirable oxidative degradation and corrosion. There is also a need in the industry to provide reagents for CO₂ capture that have an acceptable absorption rate and low energy regeneration of the reagents, such that the reagents can be recycled and reused for additional acceptable absorption of CO₂ capture. There is still further a need in the industry to provide acceptable CO₂ capture with reagents that do not result in undesirable oxidative degradation and corrosion of equipment or that are harmful to the environmental. Further, there is a need in the industry for the option of simultaneous capture and removal of all three flue gases CO₂, NOₓ and SOₓ. There is also the need in the industry for the option of combining the removal of CO₂, NOₓ and SOₓ into one step using non-toxic reagents, which would provide achieving further cost savings, making flue gas removal more economical and environment friendly. There is also a need in the industry for the utilization of captured CO₂, such as conversion of the captured CO₂ in economically viable and environmentally friendly components.

### BRIEF DESCRIPTION OF THE DRAWINGS

Subject matter hereof may be more completely understood in consideration of the following detailed description of various embodiments in connection with the accompanying figures, in which:

FIGS. 1A-1D are schematics illustrating the effects of a frothing agent adsorbing on the liquid-air interface of bubbles to reduce the surface tension and thereby decreasing the bubble size, according to certain embodiments of the present invention. FIG. 1A illustrates the effect on bubble generation without a frothing agent, while FIG. 1B illustrates the effect on bubble generation with a frothing agent (arrows in FIGS. 1A-1B indicating the flow direction of the bubbles). FIGS. 1C-1D illustrate more specifically the effect of a frothing agent on a single carbon dioxide bubble generation with the frothing agent adsorbing to the carbon dioxide bubble with the polar hydrocarbon group of the frothing agent absorbed at the gas-liquid interface and the non-polar group of the frothing agent oriented towards the gas portion of the carbon dioxide bubble.

FIG. 2 is a process flow diagram for a scrubber system comprising a scrubber assembly for CO₂ capture and a regeneration assembly for regenerating reagents and CO₂ in a purified form, the scrubber system capable of providing a continuous-loop of CO₂ capture from a gaseous feedstock using a slurry scrubbing solution in the scrubber assembly to produce a resultant product, and the regeneration assembly capable of transforming the resultant product into regenerated CO₂ in a purified form and a regenerated slurry scrubbing solution, according to certain embodiments of the present invention. The gaseous feedstock preferably comprising CO₂ mixed with air can be fed into the scrubbing column from a gas inlet, preferably proximate a bottom portion of the scrubbing column, and the slurry scrubbing solution is fed into the scrubbing column from a slurry solution inlet, preferably proximate a top portion of the scrubbing column, whereby a counter-current direction between the slurry scrubbing solution and the air flow (CO₂/air mixture) is provided within the scrubbing column. After CO₂ is absorbed by the slurry scrubbing solution (in some aspects preferably comprising a sodium carbonate solution), the resultant reactant solution (in some aspects preferably comprising a sodium bicarbonate solution) exits the scrubbing column at a resultant product outlet, preferably proximate a bottom portion of the scrubbing column. The resultant reactant solution exiting the scrubbing column can be optionally preheated by a regenerated slurry scrubbing solution (in some aspects preferably comprising a sodium carbonate solution) returning to the scrubbing column. The regenerated slurry scrubbing solution preferably returning to the scrubbing column from a flash drum, which as a result of the heat transfer between with the resultant production cools the regenerated scrubbing solution before the regenerated scrubbing solution being pumped back into the scrubbing column. The resultant reactant solution preheated by the regenerated scrubbing solution can be fed into a regenerator, preferably a flash drum proximate a feed inlet, wherein the resultant reactant solution is transformed back into the regenerated slurry scrubbing solution and regenerated CO₂ provided in purified form compared to the gaseous feedstock, whereby the regenerated slurry scrubbing solution and regenerated CO₂ can be separated from each other, with the regenerated slurry scrubbing solution capable of being recycled and reused in the scrubbing column to capture additional CO₂ from a continual flow of gaseous feedstock.

FIG. 3 is a schematic of an exemplary heat cycle loop provided by the CO₂ capture scrubber and regeneration system of FIG. 2, whereby input heat can be provided by steam (the regeneration process is omitted from the view but would take place in-line with the 98° C. stream), according to certain embodiments of the present invention.

FIG. 4 is a graph illustrating the calculated percentage of CO₂ absorbed by three different scrubbing solutions over time until steady state and a maximum absorbance was achieved using the system of FIG. 2, which included 0.2 M scrubbing solutions of (i) Na2CO3, (ii) NaOH and (iii) MEA, each scrubbing solution concentration at 38.5° C., with the error bars representing standard error, wherein in the first 3 minutes from the start of experiment the percentage of CO₂ absorbed in the instance of the scrubbing solution being MEA and NaOH rises to about 80-85% very fast and then finally reaching a maximum value of about 97% after 5 minutes, and the scrubbing solution being Na₂CO₃ the percentage of CO₂ absorbed gradually increases to about 36% in the first 3 minutes and reaches asymptote at about 55.6% after 5 minutes, indicating that the absorption efficiency of Na₂CO₃ is much less compared to the other two scrubbing solutions, according to certain embodiments of the present invention.

FIG. 5 is a graph illustrating the CO₂ absorption efficiency of a 0.2 M Na₂CO₃ scrubber solution being enhanced with a frothing agent at 10 ppm frother concentration at 38.5° C., with the error bars representing standard error, wherein in the first 2 minutes the percentage of CO₂ absorbed reaches about 93% in the instance of the frothing agent being DF200, and wherein a maximum value of about 99.9% after 5 minutes is reached in the instance of the frothing agent being DF200, DF250 or AF68, and wherein the frothing agent being AF70 or DF400 only able achieve a maximum absorbance of about 62.8%, according to certain embodiments of the present invention.

FIG. 6 is a graph illustrating the estimated CO₂ bubble size distribution in a scrubber solution comprising sodium carbonate solution with different frothing agents, whereby the estimated CO₂ bubble size in diameter and frequency count for each of the frothing agents AF68, DF200, AF70, DF400 and DF250, wherein the frothing agent DF200 provided the smallest and most uniform bubble size distribution compared to the other frothing agents, wherein the frothing agents DF250 and AF68 provided similar size distributions, and wherein the frothing agents AF70 and DF400 provide the largest bubble sizes, such that a frothing agent providing a narrow size distribution and smaller CO₂ bubble size provided more effective mass transfer area, according to certain embodiments of the present invention.

FIG. 7 is a graph illustrating the effect of frothing agent concentration in a scrubbing solution on CO₂ absorption performance, wherein concentrations of 15 ppm, 10 ppm and 5 ppm for the frothing agent DF200 were provided in 0.2 M sodium carbonate solution at 38.5° C., the error bars representing standard error, whereby the frothing agent concentrations of 10 ppm and 15 ppm showed similar absorption performance, reaching a maximum value of about 99.9%, and the frothing concentration of 5 ppm only achieving a 70.3% maximum absorbance, according to certain embodiments of the present invention.

FIG. 8 is a graph illustrating the rate of absorption of CO₂ with a scrubbing solution comprising 0.2 M Na₂CO₃ and various different frothing agents at different concentrations, with the error bars representing standard error, according to certain embodiments of the present invention.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

FIG. 9 is a process flow diagram of a system and process for continuous CO₂ capture and thermal regeneration of a scrubbing solution, wherein flue gas (CO₂ mixed with air) is fed into the scrubbing column from the bottom and the scrubbing solution is fed into the scrubbing column from the top for a counter-current direction than for CO₂ absorption by the scrubbing solution; after CO₂ is absorbed by the scrubbing solution, the resultant reactant solution (in some aspects preferably a sodium bicarbonate solution) exits the scrubbing column and is fed to a thermal regeneration system and process whereby the CO₂ and scrubbing solution are regenerated, with the scrubbing solution fed back to the scrubbing column for further CO₂ absorption from the flue gas.

FIG. 10 is a process flow diagram of a system and process for continuous CO₂ capture and regeneration of reagents and CO₂ in a purified form, wherein a gaseous feedstock, preferably a flue gas, is introduced into a CO₂ capture assembly, preferably a scrubbing assembly having a scrubbing column and a slurry scrubbing solution preferably comprising sodium hydroxide, sodium carbonate, or a mixture thereof, to produce a resultant product, preferably a sodium bicarbonate that exits the CO₂ capture assembly. Resultant product can be introduced into a regeneration assembly preferably comprising a reaction tank wherein the resultant product is reacted with an acid reagent, preferably the acid reagent comprising sulfuric acid, to regenerate CO₂ in a purified form and a resultant salt solution, the resultant salt solution preferably comprising a sodium carbonate solution. The resultant salt solution can be subjected to an EDPM assembly, wherein the resultant salt solution is subjected to electrodialysis with one or more CEM and BPM to separate acid and base as regenerated acid and regenerated base, respectively. Regenerated base can be circulated back to the scrubber as the scrubbing solution to capture additional CO₂, regenerated slurry scrubbing solution preferably comprising sodium hydroxide, sodium carbonate, or a mixture thereof. Regenerated acid can be circulated back to the regeneration assembly, preferably a reaction tank, for additional reaction with resultant product for CO₂ regeneration and resultant salt solution formation. The foregoing system and process capable of being a continuous process for continual capture of CO₂ from the gaseous feedstock to produce regenerated CO₂ in a purified form, according to certain embodiments of the present invention.

FIG. 11 is a process flow diagram of the system and process of FIG. 2 used in the Experiment section, wherein the CO₂ capture assembly comprises a scrubbing assembly having a scrubbing column and a slurry scrubbing solution comprising NaOH, and wherein the regeneration assembly comprises an acid/base reaction tank whereby the resultant product comprising sodium bicarbonate from the scrubbing assembly is reacted with an acid comprising sulfuric acid to regenerate CO₂ in a purified form and provide a resultant salt solution comprising sodium carbonate that is subjected to the EDPM assembly, wherein the resultant salt solution is subjected to electrodialysis with one or more CEM and BPM to separate acid comprising sulfuric acid and base comprising NaOH as regenerated acid and regenerated base, respectively. Regenerated base can be circulated back to the scrubber as at least a portion of the scrubbing solution to capture additional CO₂. Regenerated acid can be circulated back to the regeneration assembly as a portion of the reactive acid for additional reaction with resultant product for CO₂ regeneration and resultant salt solution formation. The foregoing system and process capable of being a continuous process for continual capture of CO₂ from the gaseous feedstock to produce regenerated CO₂ in a purified form, according to certain embodiments of the present invention.

FIGS. 12A-12B are schematics of the two-compartment configuration of electrodialysis with bipolar membrane (EDPM) separation in FIGS. 2 and 3, each compartment of the two-compartment having a bipolar membrane (BPM) and a cation exchange membrane (CEM) as the repeating unit as shown by the dotted box in FIG. 3A, and the CEM and BPM membranes of FIG. 3A shown in more detail in FIG. 3B, according to certain embodiments of the present invention.

FIG. 13 is a graph illustrating CO₂ capture efficiency of NaOH at various concentrations at 38° C., wherein the error bars represent standard error (n=3).

FIG. 14 is a graph illustrating change in acid and base concentration with time at temperature T=30° C., voltage V=18 V in the EDPM system and process, according to certain embodiments of the present invention.

FIG. 15 is a graph illustrating current density versus time until the current reached a maximum value in the EDPM system and process, wherein the error bars represent standard error from three independent measurements, according to certain embodiments of the present invention.

FIG. 16A is a graph illustrating the effect of current density on energy consumption (vertical axis on the left side) per Kg of CO₂ captured and current efficiency (vertical axis on the right side) for the EDPM system and process, according to certain embodiments of the present invention.

FIG. 16B is a graph illustrating the effect of current density on NaOH concentration and CO₂ capture efficiency for the EDPM system and process, with the error bars representing the standard error of three independent measurements, according to certain embodiments of the present invention.

FIG. 17 is a schematic of the dimensions of a pilot scale single stage absorption capture column for the simultaneous capture of CO₂, NOₓ and SOₓ from flue gas.

FIG. 18 is a graph illustrating the absorbance of CO₂ versus time in 0.2 mol/L Na₂CO₃ solution and the various concentrations of H₂O₂/NaOCl at 318K, with the error bars representing standard error (n=3).

FIG. 19 is a graph illustrating rate constant with respect to concentration for CO₂ absorbance in 0.2 mol/L Na₂CO₃ solution and an H₂O₂/NaOCl solution, with the error bars representing standard error (n=3).

FIG. 20 is a graph illustrating the absorbance of NO versus time in 0.2 mol/L Na₂CO₃ solution and various concentrations of H₂O₂ at 318K, with the error bars representing standard error (n=3).

FIG. 21 is a graph illustrating the absorbance of NO versus time in 0.2 mol/L Na₂CO₃ solution and various concentrations of NaOCl at 318K, with the error bars representing standard error (n=3).

FIG. 22 is a graph illustrating the effect of oxidizer concentration on the absorbance rate of NO at 318K.

FIG. 23 is a graph illustrating the absorbance of SO₂ versus time in 0.2 mol/L Na₂CO₃ solution and various concentrations of H₂O₂/NaOCl at 318K, with the error bars representing standard error (n=3).

FIG. 24 is a graph illustrating the absorbance of CO₂, NO and SO₂ versus pH with 750 μ/L H₂O₂ concentration at 318K at 5 minute intervals, with the error bars representing standard error (n=3).

FIG. 25(a) is a schematic of a membrane electrolysis cell for converting CO₂ to oxalate, according to certain embodiments of the present invention.

FIG. 25(b) is a schematic of a membrane electrolysis cell for converting CO₂ to oxalate, whereby the cathode is wrapped around the anode in a cylindrical configuration and the cathode having an outer coating, according to certain embodiments of the present invention.

FIG. 26 is an XRD analysis of an oxalate sample produced according to certain embodiments of the present invention.

FIG. 27 is a schematic of a CO₂ capture and electrochemical reduction loop, which the captured CO₂ is illustrated to produce an oxalate salt as an intermediate to oxalic acid, according to certain embodiments of the present invention.

FIG. 28 is a schematic flow diagram illustrating CO₂ capture with EDBM separation, such as shown in more detail in FIG. 11, and simultaneous captured CO₂ utilization in an electrochemical reduction loop as shown in FIG. 27 for the conversion of captured CO₂ to oxalic acid, which may be utilized to selectively precipitate rare earth minerals from a feedstock, such as in neutralizing red mud (bauxite residue from alumina production) alkalinity providing neutralized red-mud, which allows for the use of the red mud as a useful material, such as employed as a catalyst or catalyst support, and/or the recovery of the rare earth elements from the red-mud, such as Al, Na, Fe and Ti, according to certain embodiments of the present invention.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

The present disclosure is generally directed at the capture of pollutants. In some aspects, the present disclosure is directed at the capture of CO₂, particularly CO₂ emissions. In some other aspects, the present disclosure is directed at the simultaneous capture of two or more emissions. In some preferred aspects, the present disclosure is directed at the simultaneous capture of two or more of CO₂, NOₓ and SOₓ emissions. In some preferred aspects, the present disclosure is directed at the simultaneous capture of CO₂, NOₓ and SOₓ emissions.

The present disclosure is also generally directed at the utilization of captured pollutants. In some aspects, the present disclosure is directed at the utilization of captured CO₂, particularly captured CO₂ emissions, more preferably captured CO₂ emissions that have been cleaned or purified. In some other aspects, the present disclosure is directed at the utilization of at least one captured emission from the simultaneous capture of two or more emissions. In some preferred aspects, the present disclosure is directed at the utilization of at least one captured emission from the simultaneous capture of two or more emissions of CO₂, NOₓ and SOₓ. In some preferred aspects, the present disclosure is directed at the utilization of captured emissions from the simultaneous capture of CO₂, NOₓ and SOₓ emissions.

International Application PCT/US23/11646 discloses the capture of contaminants from flue gas using an absorption column, particularly the chemical absorption capture of CO₂ using a scrubbing absorption column containing a slurry solution having a frothing agent, the contents of which are incorporated-by-reference in their entirety herein.

International Application PCT/US23/11818 discloses carbon dioxide capture and the regeneration of reagents relating to the carbon dioxide capture, the contents of which are incorporated-by-reference in their entirety herein.

International Application PCT/US22/51623 discloses the capture of contaminants from flue gas using an absorption column, particularly a single wet scrubbing absorption column at alkaline pH conditions for the simultaneous capture of CO₂, NOₓ and SOₓ from flue gas and methods of simultaneously capturing CO₂, NOₓ and SOₓ from flue gas, the contents of which are incorporated-by-reference in their entirety herein.

The term “slurry solution” or “slurry scrubbing solution” as used herein refers to a liquid-solid fluid mixture with a specific gravity greater than 1.

The term “frother” or “frothing agent” as used herein refers to a reagent used to control the size and stability of one or more gas bubbles in a liquid, preferably the bubbles comprising air and/or CO₂. In some instances, a “frother” or “frothing agent” is an organic heteropolar compound, such as an alcohol or polyglycol ether, that due to its heteropolar nature absorbs at the gas/liquid interface and as a result, lowers the surface tension, which has the effect of producing smaller bubbles than the bubbles produced in the absence of the “frother” or “frothing agent”. In some preferable aspects, the “frother” or “frothing agent” minimizes or prevents bubble coalescence, which minimizes bubbles from becoming bigger and thereby producing uniformly small sized bubbles.

The term “clean CO₂” or “purified CO₂” as used herein refers to a CO₂ stream that is substantially devoid of impurities, such as sulfur oxides, nitrogen oxides, oxygen, carbon monoxide and water, such that the CO₂ purity is at least industrial or medical grade with a 99.5% purity.

The present disclosure is further generally directed towards the utilization of captured CO₂ in a purified form as a feedstock for the generation of one or more other chemicals. In some aspects, the one or more other chemicals is generated by an electrochemical reduction process. In some aspects, the one or more other chemicals generated by the electrochemical reduction process comprises an oxalate salt, which can be converted to oxalic acid.

The present disclosure is further generally directed to the utilization of captured CO₂ in a purified form as an acid source for neutralization of red mud. The present disclosure is further generally directed to the utilization of oxalic acid generated from captured CO₂ as an acid source for neutralization of red mud. The present disclosure is further generally directed to the utilization of oxalic acid generated from captured CO₂ as a solvent utilized in the extraction of rare earth minerals from red mud and/or neutralized red mud.

### I. Use of Frothers to Improve the Absorption Efficiency of Dilute Sodium Carbonate Slurry for Post Combustion CO₂ Capture

With current environmental regulations, CO₂ capture is very crucial for the survival of fossil-fuel power plants, particularly coal-fired power plants in the near future. The present inventors investigated CO₂ absorption performances of Na₂CO₃, NaOH, Monoethanolamine (MEA) and frother-enhanced Na₂CO₃ slurry solutions in a gas-liquid countercurrent column. A frothing agent was added to the slurry solution comprising sodium carbonate in order to increase the surface area available for CO₂ transport within the packed bed. Generally speaking, the presence of the frothing agent in the slurry solution comprising sodium carbonate increased the CO₂ capture efficiency of dilute sodium carbonate slurry from 55.6% to 99.9%, before reaching saturation.

Without wishing to be bound by theory, it is believed that increasing the mass transfer kinetics by the addition of a frothing agent to the slurry solution increased the CO₂ absorption efficiency of sodium carbonate. The enhancement of the CO₂ absorption efficiency by the frothing agent was dramatic, with the increased efficiency provided by the frothing agent allowing even dilute sodium carbonate solutions to achieve greater than 80%, in some aspects greater than 85%, in some aspects greater than 90%, in some aspects greater than 95%, in some aspects greater than 97.5%, in some aspects greater than 98%, in some aspect greater than 98.5%, in some aspects greater than 99.0%, in some aspects greater than 99.5%, in some aspect greater than 99.75%, and in some aspects up to 99.9%, CO₂ capture.

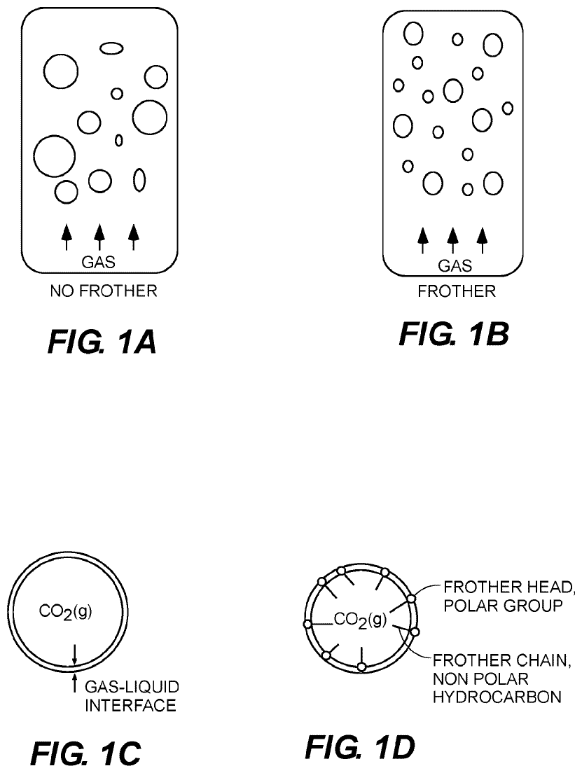

Frothers or frothing agents are surfactants that adsorb on the liquid-air interface of the bubbles, reducing the surface tension and thereby decreasing the bubble size as shown in FIGS. 1A-1D. The CO₂ absorption rate of sodium carbonate is low compared to NaOH and MEA, which is believed to be due to limited kinetics from the low concentration of CO₂ in aqueous solution. The present inventors have discovered that one way to overcome this obstacle is to increase the rate of physical mass transfer, which can be achieved by creating smaller and uniform bubbles. This decrease in size increases the interfacial interaction area between gas and liquid increasing the mass transfer rate and allowing more gas to be absorbed faster.

Bubble size can be influenced by adding surfactants known as frothers. Frothers or frothing agents can prevent bubble coalescence, which stops the bubbles from becoming bigger and thereby producing tiny and uniform bubbles. Frothing agents have a polar hydrocarbon group and a non-polar group, with the non-polar group being oriented towards the air and the polar group adsorbed at the air-liquid interface as shown in FIG. 1D.

The present inventors have discovered an additive that will increase the absorption rate of a slurry solution comprising sodium carbonate and have minimal or no effect on the energy required for reagent regeneration. In some preferred aspects, the additive is a frother or frothing agent. In some other preferred aspects, the frothing agent is provided in a minimal concentration, such that the reagent cost of sodium carbonate and the additive is commercially acceptable.

The frothing agent can be utilized in a scrubber system according to certain aspects of the present invention, the scrubber system comprising a scrubbing column having a top end and a bottom end, wherein the scrubbing column comprises a slurry solution, wherein a gaseous mixture comprising carbon dioxide is fed into the bottom end of the scrubbing column, and wherein the slurry solution comprises at least one frothing agent.

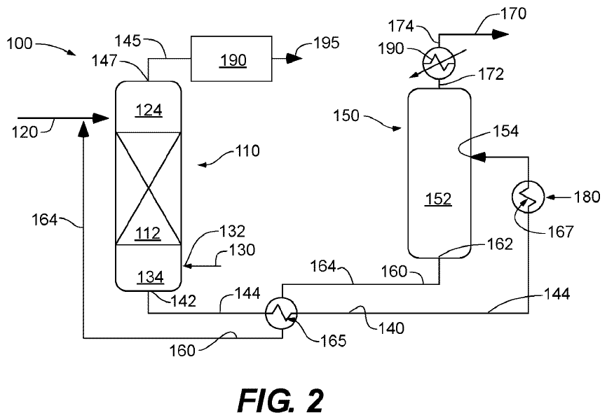

In certain aspects, a frothing agent can be utilized in a scrubber system, such as a scrubbing column as shown in FIG. 2. The scrubber system preferably comprises a scrubbing column having a top end and a bottom end, wherein the scrubbing column comprises a slurry solution, wherein a gaseous mixture comprising carbon dioxide is fed into the bottom end of the scrubbing column, and wherein the slurry solution comprises at least one frothing agent.

In some aspects, the frothing agent comprises at least one compound of Formula (I):

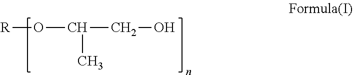

wherein R is H or CH₃, and wherein n is greater than 2 and up to 34, preferably n being between 3 and 34, more preferably n being between 3 and 8.

The frothing agent preferably comprises at least one compound of Formula (I), wherein the molecular weight (g/mol) is less than about 400, in some aspects less than about 390, in some aspects less than about 380, in some aspects less than about 370, in some aspects less than about 360, in some aspects less than about 350, in some aspects less than about 340, in some aspects less than about 330, in some aspects less than about 320, in some aspects less than about 310, in some aspects less than about 300, in some aspects less than about 290, in some aspects less than about 280, in some aspects less than about 270, in some aspects less than about 260, and in some aspects less than about 250.

In some aspects, the frothing agent comprises at least one compound of Formula (I), wherein the molecular weight (g/mol) is greater than 200 and less than about 400, in some aspects is greater than 200 and less than about 390, in some aspects is greater than 200 and less than about 380, in some aspects is greater than 200 and less than about 370, in some aspects is greater than 200 and less than about 360, in some aspects is greater than 200 and less than about 350, in some aspects is greater than 200 and less than about 340, in some aspects is greater than 200 and less than about 330, in some aspects is greater than 200 and less than about 320, in some aspects is greater than 200 and less than about 310, in some aspects is greater than 200 and less than about 300, in some aspects is greater than 200 and less than about 290, in some aspects is greater than 200 and less than about 280, in some aspects is greater than 200 and less than about 270, in some aspects is greater than 200 and less than about 260, and in some aspects is greater than 200 and less than about 250.

In some aspects, the frothing agent comprises at least one poly glycol ether (PEG)-based compound.

In some aspects, the frothing agent comprises at least one PEG-based compound chosen from the group consisting of CH₃ (C₃H₆O)₃OH, CH₃ (C₃H₆O)₄OH, CH₃ (C₃H₆O)_{6.3}OH, CH₃ (C₃H₆O)₃OH, H(C₃H₆O)_{6.5}OH, H(C₃H₆O)₆OH, CH₃ (C₃H₆O)₄OH(C₄H₆O), H(C₃H₆O)_{12.8}OH, H(C₃H₆O)_{16.5}OH, H(C₃H₆O)₃₄OH and mixtures thereof.

In some aspects, the frothing agent comprises at least one PEG-based compound chosen from the group consisting of CH₃ (C₃H₆O)₃OH, CH₃ (C₃H₆O)₄OH, and combinations thereof.

In some aspects, the frothing agent comprises at least one PEG-based compound, wherein the PEG-based compound is capable of producing an average bubble diameter size in a slurry solution of less than 1.8 mm, preferably less than 1.7 mm, preferably less than 1.6 mm, more preferably less than 1.5 mm, preferably less than 1.4 mm, and even more preferably less than 1.4 mm.

In some aspects, the frothing agent is capable of producing an average bubble diameter size in a slurry solution between about 0.9 mm up to about 2.0 mm, preferably between about 1.0 mm up to about 1.9 mm, preferably between about 1.0 mm up to about 1.8 mm, preferably between about 1.0 mm up to about 1.7 mm, preferably between about 1.0 mm up to about 1.6 mm, preferably between about 1.0 mm up to about 1.5 mm, preferably between about 1.0 mm up to about 1.4 mm, preferably between about 1.0 mm up to about 1.3 mm, and even more preferably between about 1.0 mm up to about 1.2 mm.

The frothing agent is preferably present in the slurry solution in an amount greater than 0 ppm up to about 40 ppm, preferably between about 1 ppm and about 35 ppm, preferably between about 2 ppm and about 30 ppm, more preferably between about 3 ppm and about 25 ppm, and even more preferably between about 5 ppm and about 20 ppm.

The frothing agent preferably comprises at least one PEG-based compound having a molecular weight (g/mol) less than about 400, in some aspects less than about 390, in some aspects less than about 380, in some aspects less than about 370, in some aspects less than about 360, in some aspects less than about 350, in some aspects less than about 340, in some aspects less than about 330, in some aspects less than about 320, in some aspects less than about 310, in some aspects less than about 300, in some aspects less than about 290, in some aspects less than about 280, in some aspects less than about 270, in some aspects less than about 260, and in some aspects less than about 250.

In some aspects, the frothing agent preferably comprises at least one PEG-based compound having a molecular weight (g/mol) greater than 200 and less than about 400, in some aspects is greater than 200 and less than about 390, in some aspects is greater than 200 and less than about 380, in some aspects is greater than 200 and less than about 370, in some aspects is greater than 200 and less than about 360, in some aspects is greater than 200 and less than about 350, in some aspects is greater than 200 and less than about 340, in some aspects is greater than 200 and less than about 330, in some aspects is greater than 200 and less than about 320, in some aspects is greater than 200 and less than about 310, in some aspects is greater than 200 and less than about 300, in some aspects is greater than 200 and less than about 290, in some aspects is greater than 200 and less than about 280, in some aspects is greater than 200 and less than about 270, in some aspects is greater than 200 and less than about 260, and in some aspects is greater than 200 and less than about 250.

Besides the frothing agent, the slurry solution further preferably comprises sodium carbonate.

As shown by the process flow diagram in FIG. 2, scrubber system 100 generally comprises scrubber assembly 110 and optionally a regeneration assembly 150. Scrubber assembly 110 preferably comprises scrubbing column 112, which contains slurry scrubbing solution 120 and gaseous feedstock 130. Slurry scrubbing solution 120 is preferably fed into scrubbing column 112 proximate a slurry solution inlet 122, which is preferably proximately located a top portion 124 of scrubbing column 112. Slurry scrubbing solution 120 can comprise fresh slurry scrubbing solution, regenerated slurry scrubbing solution, or a mixture thereof. Gaseous feedstock 130 preferably comprises a mixture of CO₂ and air, which is preferably fed into scrubbing column 112 proximate a gas inlet 132, which is preferably proximately located a bottom portion 134 of scrubbing column 112. Scrubbing column 112 is preferably a packed-bed counter-current absorption column, such that the flow of slurry scrubbing solution 120 is in an opposite direction to the flow of gaseous feedstock 130.

Slurry scrubbing solution 120 and gaseous feedstock 130 are each preferably fed into scrubbing column 112, such that scrubber assembly 110 is capable of providing continuous CO₂ capture. During normal operation, CO₂ is absorbed from gaseous feedstock 130 by slurry scrubbing solution 120 providing resultant product 140, which is preferably a resultant product solution, configured to exit scrubbing column 112 proximate at a resultant product outlet 142, preferably proximately located bottom portion 134 of scrubbing column 112, providing resultant product stream 144. Resultant product 140 having exited scrubbing column 112 can optionally be subjected to one or more further processing processes.

For instance, resultant product 140 can be subjected to an optional heat transfer process. In one instance, resultant product stream 144 can be preheated by regenerated scrubbing solution stream 164 in a heat exchanger 165, wherein regenerated scrubbing solution stream 164 returning to scrubber assembly 110 prior to regenerated scrubbing solution 160 being fed back into scrubber column 112 via scrubber slurry solution inlet 122. Regenerated scrubbing solution stream 164 can have a higher temperature than resultant product stream 144, such that heat exchange between resultant product stream 144 and regenerated scrubbing solution stream 164 can cool a temperature of the regenerated scrubbing solution stream 164 before regenerated scrubbing solution 160 is fed back into scrubbing column 112. In another instance, resultant product 140 can be heated in via heat exchanger 167 by heat source 180. Preferably, heat source 180 is steam (e.g., 30 psi steam) such as to raise the temperature of resultant product 140 to an increased temperature, such as to a temperature greater than 90° C., in some aspects greater than 95° C., in some aspects greater than 96° C., in some aspects up to about 98° C., although other sources of heat can be used to heat resultant product 140. In some preferred aspects, resultant product 140 is preheated one or more times prior to being fed into regeneration assembly 150.

Resultant product 140 can be subjected to an optional regeneration process. Resultant product stream 144 can be fed into regeneration assembly 150 comprising a regeneration vessel 152 via a regeneration feed inlet 154. Regeneration vessel 152 preferably comprises a flash drum. Once resultant product stream 144 is fed into regeneration vessel 152, resultant product 140 is transformed into regenerated slurry scrubbing solution 160 and regenerated CO₂ 170, which can be separated from each other. Regenerated CO₂ 170 can exit regeneration vessel 152 via a gas outlet 172, preferably providing a continuous regenerated CO₂ stream 174, which is preferably a purified form of CO₂. Regenerated slurry scrubbing solution 160 can exit regeneration vessel 152 via regeneration solution outlet 162, providing regenerated scrubbing solution stream 164. Regenerated scrubbing solution stream 164 is preferably recycled back to scrubbing assembly 110 and reused as a slurry scrubbing solution for capture of additional CO₂.

As provided by the foregoing disclosure in relation to FIG. 2, the scrubbing assembly 110 and regeneration assembly 150 can provide a continuous loop between resultant product 140 being transformed into regenerated slurry scrubbing solution 160, such that the input gaseous feedstock 130 is converted into regenerated CO₂ 170 as an output, which can be subjected to a condenser 190, which condenses the regenerated CO₂ 170 in a purified form to provide condensed CO₂ in a purified form. Another output comprises outlet gas stream 145 having CO₂ absorbed within scrubbing column 112, such that gas stream 145 exits scrubbing column via gas outlet 147. Outlet gas stream 145 is preferably cleaned with respect to CO₂, which can be subjected to gas analysis by a gas analyzer 190, and removed into the atmosphere as cleaned exhaust 195.

In some aspects, slurry scrubbing solution 120 comprises sodium carbonate, such that the scrubber system reacts the sodium carbonate of the slurry solution with the CO₂ of the gaseous mixture to provide a resultant product preferably comprising a sodium bicarbonate solution. In some preferred aspects, regenerated slurry scrubbing solution 160 comprises sodium carbonate, such that the scrubber system reacts the sodium carbonate of the regenerated slurry solution with the CO₂ of the gaseous mixture to provide a resultant product preferably comprising a sodium bicarbonate solution.

In some aspects, the sodium bicarbonate solution produced in the scrubbing column can be subjected to a regeneration process to provide a regenerated sodium carbonate solution and a regenerated carbon dioxide, which is provided in a purified form compared to the CO₂ of the gaseous mixture introduced into the scrubbing column. In some aspects, the regenerated carbon dioxide is essentially pure. In some aspects, the regenerated carbon dioxide is a gas or liquid. In some aspects, the regenerated carbon dioxide has a purity greater than 95%, in some aspects greater than 97%, in some aspects greater than 98%, in some aspects greater than 99%, in some preferably aspects greater than 99.5%, and in some more preferable aspects greater than 99.9%.

In some preferred aspects, the resultant product is separated into a regenerated slurry solution and a regenerated carbon dioxide, preferably within a flash drum. In some preferred aspects, the regenerated slurry solution comprises a sodium carbonate solution. The regenerated slurry solution can be recycled, such that it is cycled back through the scrubbing column to provide further capture of CO₂ from a gaseous feedstock introduced into the scrubbing column. In some aspects, the regenerated slurry solution, such as a regenerated sodium carbonate solution, is capable of reacting with the CO₂ of the gaseous mixture fed into the scrubber column to provide a second resultant product, such as a second sodium bicarbonate solution.

In some aspects, the regenerated sodium carbonate solution fed to the scrubbing column, reaction of the CO₂ of the gaseous mixture fed into the scrubber column with the regenerated sodium carbonate solution to provide a sodium bicarbonate solution, and the sodium bicarbonate solution being subjected to a regeneration process to provide a second regenerated sodium carbonate solution that is separated from the regenerated CO₂, can be provided as a continuous process.

In the continuous process, the gaseous mixture fed into the scrubbing solution can be consumed within the scrubbing column by reacting the slurry scrubber solution with CO₂ of the gaseous mixture to produce a resultant product, thereby allowing other air components to vent out of the scrubbing column. The resultant product can be further processed to produce purified CO₂ and regenerate the slurry scrubbing solution for additional use within the scrubbing column. For instance, any regenerated sodium carbonate solution fed back to the scrubbing column allows for additional CO₂ capture to provide a resultant sodium bicarbonate, which can be subjected to processing for regeneration of a subsequent regenerated sodium carbonate solution and purified CO₂, which can be a continuous process comprising one or more regeneration cycles.

In some aspects, the regenerated slurry solution, preferably comprising a sodium carbonate solution, comprises at least a portion of the frothing agent concentration. The frothing agent concentration may need to be replenished after a certain number of regeneration cycles. In some aspects, the frothing agent concentration is replenished in the sodium carbonate solution with a fresh aliquot of frothing agent after about 2 to about 10 cycles, in some aspects after about 2 to about 6 cycles, and in some aspects between about 3 and about 4 cycles.

In some aspects, the slurry solution and/or the regenerated slurry solution comprising one or more frothing agents providing CO₂ capture of at least 90%, in some aspects at least 95%, in some aspects at least 98%, in some aspects at least 99%, in some aspects at least 99.5%, and in some aspects at least 99.9%.

### EXPERIMENTAL

### Column Properties

A CO₂ capture system was designed and built as shown in FIG. 2 having a scrubbing column with a gas inlet, resultant product outlet, and gas outlet, whereby the result product outlet is shown as being in fluid communication with a means for regeneration of a slurry solution and CO₂, such as a flash drum. The CO₂ capture system also can have a heat recycle loop in fluid communication between the scrubbing column and the means for regeneration, a condenser in fluid communication with the means for regeneration, and a heat inlet into the system.

As it relates to the scrubber column, polypropylene pall rings (1.2 cm×1.2 cm) were used as packing in the scrubber column. The height of a packed bed scrubbing column (Z) was calculated using the contact tower design equation of Equation (1).

$$Z = \frac{{G}_{s}}{{K}_{y} * a} {\int }_{{Y}_{2}}^{{Y}_{1}} \frac{dY}{Y - {Y}^{*}} \qquad ( 1 )$$

where Gs represents molar flow of solute-free gas per cross-sectional area of the column, a is the interfacial area available for mass transport, Ky accounts for overall gas phase mass transfer coefficient, Y is the fraction of moles of gas phase solute per moles of solute-free gas, and Y* denotes the gas phase mole fraction in equilibrium with the liquid phase. The denominator of the integral represents the driving force for mass transfer and is integrated over the condition of the gas phase from the top to the bottom of the column.

Given that the interfacial area a is in the denominator of the design equation, it is advantageous to have a large amount of interfacial area within the scrubbing column.

### Materials and Methods

CO₂ Absorption Experiments without Frother Addition

The mini-pilot scale setup shown in FIG. 2 was used to conduct experiments on percentage of CO₂ absorbed with sodium carbonate and other reagents. The packed-bed absorption column (Height: 274.3 cm, Diameter: 10.16 cm; Packing: Polypropylene pall rings 1.2 cm×1.2 cm; Packed bed height: 121.92 cm) shown on the left side in FIG. 2 is used as a counter current absorption column. The top portion of the capture column (213.36 cm) is made of see through polyacrylic plastic and the bottom portion is made of steel to ensure robustness. For the absorption experiments Na₂CO₃ (99.8% pure) was obtained from Duda Energy while NaOH (99%) and MEA (reagent grade) were obtained from Sigma-Aldrich. The CO₂ gas cylinders (99% pure) were obtained from Grainger. In order to simulate the flue gas, a gaseous mixture containing 16% by volume CO₂ and rest air was continuously fed into the bottom of the scrubbing column with the help of a gas diffuser. Gas flow rate was maintained at 21 LPM. Separate flow meters were installed for CO₂ and air to measure the volumetric flow and to control the percentage of CO₂ in the gas stream. CO₂ and air flow rates were measured with gas flow meters (OMEGA) equipped with gas controllers (McMaster-Carr). One of ordinary skill will appreciate that this gaseous mixture inlet could be replaced with the actual flue gas of a power plant.

The percentage CO₂ of the simulated flue gas exiting out from the top of the column was measured with Quantek Model 906 infrared gas analyzer calibrated with a 20-vol % CO₂/N₂ reference gas. Several flow rates (3-10 Liters per minute) were tested for the aqueous solutions of Na₂CO₃, NaOH and MEA. The data on percentage of CO₂ absorbed was continuously recorded by the data logger connected to the gas analyzer. After each experiment the data logger was connected to the computer and the graph generated from it was integrated to calculate the total moles of CO₂ absorbed per minute. The accuracy of the data was ensured by repeating these experiments in triplicates. For a 16% CO₂ gas stream (simulating a power plant flue gas) the optimum parameters were found to be: 0.2 M sodium carbonate solution at 7.5 Liters per minute flow rate. The effect of temperature on absorption efficiency was also studied by heating the scrubbing solution with immersion tank heater to vary the temperature of the Na₂CO₃ solution from 25° C. to 60° C. The CO₂ was regenerated along with the scrubbing solution as shown in FIG. 2 and was again recycled through the scrubbing column. Table 2 shows the typical operating conditions for the CO₂ scrubbing and regeneration setup.

TABLE 2
Typical operating parameters for the
scrubbing setup shown in FIG. 2.

| Experimental Conditions | | |
|---|---|---|
| Scrubbing solution flowrate | 7.5 | L/min |
| Gas inlet temperature | 31° | C. |
| Scrubbing solution inlet temperature | 38.5° | C. |
| Column operating pressure | 101.325 | kPa |
| Liquid/gas-ratio (Kg/Kg) | 4.3 | |
| Gas composition | 16% vol CO₂, rest air | |
| Desorption temperature | 98° | C. |

CO₂ Absorption Experiments with Frother Addition

A frothing agent was added in trace amounts to Na₂CO₃ slurry scrubbing solutions to create small and uniform bubbles when air was introduced into the liquid solution. Several different frothing agents were tested at varying concentrations from 5 ppm to 20 ppm at 5 ppm increments, as provided in Table 3. Absorption efficiency of frother-modified sodium carbonate solution was recorded at regular intervals of time. The frothers were obtained from Cytec Solvay group. Although prices of most of these frothers are unknown, it was estimated from known sources that the frothing agent price was around 1.2-1.4$/Kg.

TABLE 3
Type of frothing agent used and related properties.

| Frothing | | Frother | Molecular | Molecular |
|---|---|---|---|---|
| Agent | Manufacturer | Type | Formula | Weight |
| DF200 | DOW Chemical | Polyglycol | CH₃(C₃H₆O)₃OH | 206.29 |
| DF250 | DOW Chemical | Polyglycol | CH₃(C₃H₆O)₄OH | 206.29 |
| DF400 | DOW Chemical | Polyglycol | H(C₃H₆O)_{6.5}OH | 206.29 |
| AF68 | Solvay | Polyglycol | (mixture) n/a | — |
| AF70 | Solvay | Alcohol | (CH₃)₂CHCH₂CHOHCH₃ | 102.17 |

### Reagent Regeneration and Heat Duty

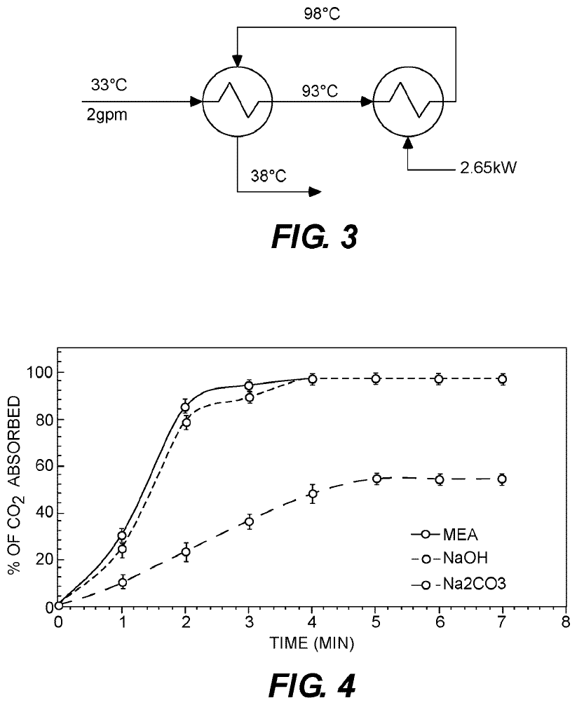

The means for regeneration can comprise a reagent regeneration setup (CO₂ stripper) consisting of a series of heat exchangers accompanied by a 19 Liter flash drum and a condenser. An exemplary overall heat recycle loop is provided in FIG. 3. The waste heat is reused with the help of heat exchangers for the thermal regeneration setup. Looking at the regeneration system energetically, the heat required to heat the inlet is already present in the outlet, such that the heat that needs to be added should be no more than required to make up the heat lost due to entropy. This should allow a significant reduction in the energy cost from 90 kWhr/m3 to about 3 kWhr/m3 to about 7 kWhr/m3. The total regeneration energy is calculated based on energy provided and also enthalpy change (ΔH) of the reagent used.

### Experimental Procedure

The setup shown in FIG. 2 was used to conduct continuous CO₂ capture and regeneration experiments. The experiment was started by turning the gas on with 16% volume CO₂ with the remaining comprising air in order to simulate flue gas. Once the gas analyzer started recording the CO₂ data, a sodium carbonate solution from a reserve tank (now shown-100 Liter) was pumped to the top of the scrubbing column as the slurry solution at 7.5 Liters per minute flow rate, and CO₂ absorption data was continuously recorded by data logger on the gas analyzer. After 5 minutes from start, the CO₂ absorption reached steady state, and then the bicarbonate solution coming out of the scrubbing column deposited in the bicarbonate reserve tank (not shown) was sent through the desorption setup for regeneration and the desorbed solution (e.g., regenerated sodium carbonate solution) was pumped back into the sodium carbonate reserve tank. The entire CO₂ absorption and desorption setup was then continuously run for 2 hours to ensure no discrepancy. The CO₂ absorption data was continuously recorded by the gas analyzer for 2 hours and no decrease in absorption rate was observed for the entire experiment. Each experiment was repeated three times to ensure reproducibility.

### Results and Discussion

The gas analyzer continuously measured the percentage concentration of the CO₂ being fed into and exiting out from the top of the scrubbing column. The absorption efficiency of CO₂ (as % of CO₂ absorbed) was calculated by Equation (2):

$$Absorption efficiency ( or ) \% of CO absorbed = \frac{{X}_{in} - {X}_{out}}{{X}_{in}} \times 100 \qquad ( 2 )$$

where Xᵢₙ is number of moles of the gas going into the scrubbing column and Xₒᵤₜ is number of moles of the gas coming out of the scrubbing column.

Initial experiments were conducted on Na₂CO₃ solution as the scrubbing slurry solution, without the addition of any frother to compare the CO₂ absorption efficiency of Na₂CO₃ with that of MEA and NaOH as the scrubbing solutions. Later, various frothers were added to the Na₂CO₃ scrubbing solution at 5 ppm incremental concentrations. Adding a frothing agent improved the absorption efficiency of Na₂CO₃ solution from 55.6% to 99.9%. The 99.9% absorption efficiency removal is based on 0.05 to 0.1% instrument error of the gas analyzer. Based on the work of Mai et al. on vapor-liquid equilibria for carbonate-bicarbonate-water-CO₂ system at 101 kPa and 38° C., the experiments stayed on the lower end of the sodium carbonate concentrations (0.1-0.3 mol/L) for conducting CO₂ absorption. Depending on the molar ratio of CO₂ converted and sodium carbonate (0.2 mol/L) used, the fraction of sodium carbonate converted to bicarbonate is only 0.46 without the frothing agent, which is believed to be due to slower absorption kinetics. After the addition of the respective frothing agent, the conversion increased to 0.81, which corresponds to a 43.2% increase.

The experimental uncertainty was calculated and error bars were plotted within the 95% confidence interval for all the experiments. These results are discussed in detail below.

Absorption Results without the Addition of a Frothing Agent

Based on Equation (5) above, the percentage of CO₂ absorbed was calculated from start of the experiment until it reached steady state and a maximum absorbance as shown in FIG. 4. Initial experiments were conducted with NaOH, monoethanolamine (MEA) and Na₂CO₃ on the scrubbing column of FIG. 2 to compare the reagents for CO₂ capture efficiency. Experimental results suggest that the absorption efficiency of amines and NaOH are almost the same, while the absorption efficiency of Na₂CO₃ is much less compared to the other two. The absorption efficiency of these reagents were noted at a concentration of 0.1 M, 0.2 M and 0.3 M in water, with 2-3% uncertainty.

The percentage of CO₂ absorbed at 0.1 M concentration for NaOH and MEA was almost the same at about 95% absorption efficiency, but the absorption efficiency for Na₂CO₃ was only between about 30% and about 40%. The curves in FIG. 4 show the percentage of CO₂ absorbance for all three reagents MEA (top), NaOH (middle) and Na₂CO₃ (bottom), each reagent solution at 0.2 M concentration. With the increase in concentration from 0.1 M to 0.2 M, the percentage of CO₂ absorbed for NaOH and MEA from 95% to increased about 97%, and the absorption efficiency for Na₂CO₃ increased from about 40% to about 55.6%. Each of the three reagents as slurry solutions were also tested at 0.3 M concentration in solution, but no further increase in absorption was observed.

### Effect of Temperature on Absorption Efficiency

Previous studies suggest that a temperature range of 30° C. to 40° C. is optimum for CO₂ absorption with sodium carbonate slurry. Studies conducted at MTU indicate that with increased temperature, the absorption rate of CO₂ decreases.

As the temperature was increased from 25° C. to 60° C., the rate of absorption of CO₂ decreased by 55%. It is believed that the reason for decrease in absorption efficiency at higher temperatures is because of decrease in gas solubility at elevated temperatures. Van′t Hoff's equation, which is provided in Equation (3) is believed to explain the effect of temperature on the solubility of gas.

$${k}_{H} = {k}_{H} ° exp [ \frac{- Δ {H}_{solution}}{R} ( \frac{1}{T} - \frac{1}{T °} ) ] \qquad ( 3 )$$

$$C = {k}_{H} \times ( P ) \qquad ( 4 )$$

k_{H}°—Henry's coefficient units—mol/L atm
ΔHₛₒₗᵤₜᵢₒₙ—Enthalpy of the solution units—Joules/mol
T—Slurry temperature units—K
R—Universal gas constant units—Joules/mol K
T°—298K
C—CO₂ Concentration in the solution units—mol/L
P—Partial pressure of CO₂ in gas phase units—atm
k_{H}—Henry's Law constant units—mol/L atm

With increase in temperature, from Equation (3), k_{H} should decrease. Hence, according to Henry's Law (Equation (4)), the dissolved CO₂ in solution will decrease. Therefore, the rate of CO₂ absorption decreases at higher temperatures. The optimum temperature was observed to be around 30° C. to 39° C.

Addition of Frothers for Improving Rate of Absorption of Na₂CO₃ Slurry

Without wishing to be bound by theory, it is believed that adding a frothing agent modifies the bubble surface of the absorbent solution when gas is introduced. A frothing agent generates smaller and more uniform bubble sizes, which increases the surface area of contact between the gas and liquid improving mass transfer. This improves the absorption efficiency of the scrubbing solution significantly.

The rate at which CO₂ is absorbed into carbonate solutions can be described as provided in Equation (5):

$${R}_{CO 2} = - \frac{d [ {CO}_{2} ]}{dt} = {k}_{L} a ( {c}^{*} - c ) = k [ {CO}_{2} ] \qquad ( 5 )$$

where k_{L} is mass transfer coefficient and k is the rate constant assuming first order kinetics. Rate of absorption of CO₂ is proportional to gas liquid interfacial area a, as shown in Equation (8). Increasing the interfacial area available for mass transport is advantageous for a scrubbing solution with slower absorption kinetics. The addition of a frothing agent to the scrubbing solution allows a stable bed of small bubbles to form within the column, increasing the area of gas-liquid interface within the column. This effectively makes up for the low CO₂ absorption rate of sodium carbonate slurry.

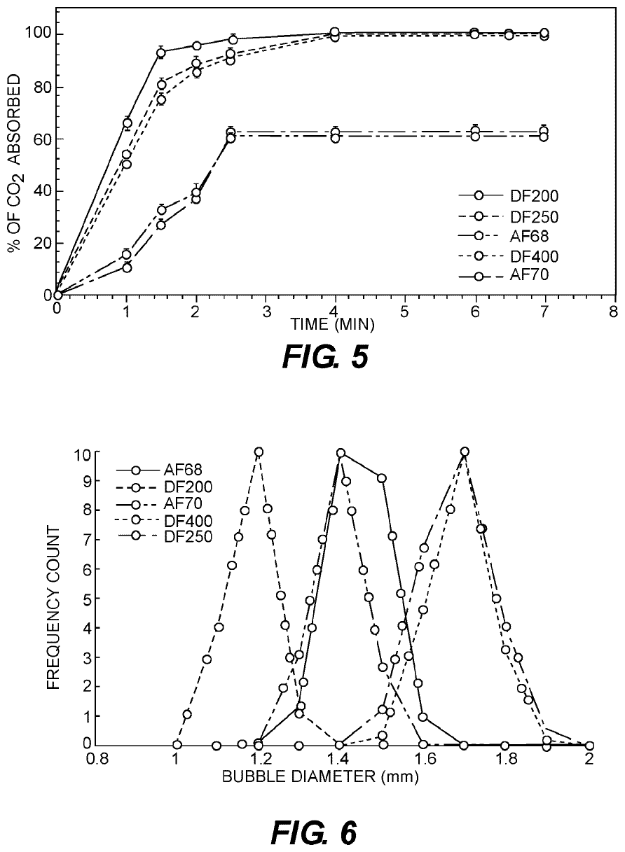

The data provided in FIG. 5 clearly shows that enhancing the sodium carbonate solution with an appropriate frothing agent greatly increases CO₂ absorption efficiency. The percentage of CO₂ absorbed was recorded from start of the experiment and was continued to be recorded after reaching steady state as well. Initially it took time for the bubbles to develop, but after reaching steady state the process was continuous. The frother-enhanced sodium carbonate solution was able to increase the CO₂ absorption efficiency of sodium carbonate solution from about 55.6% to 99.9%, which is greater than the absorption performance achieved by NaOH and MEA. FIG. 5 illustrates that the percentage of CO₂ absorbed reaches 99.9% with the frothing agent being DF200, DF250, and AF68. The frothing agents DF400 and AF70 were only able to increase the percentage of CO₂ absorbed from about 55.6% to about 62.8%. It is believed that these lower percentage of CO₂ absorbance can be attributed to the frothing agent AF70 (methyl isobutyl carbinol (MIBC)) is a weak frother, and that the frothing agent DF400 produces larger bubbles compared to the other polyglycol ether frothers (polyglycols). The effect of bubble size on CO₂ absorbance is discussed below.

### Bubble Size Analysis

Pictures of the bubbles in the column were captured using a digital video camera (Sony Alpha A7 ll). The column was illuminated to avoid unnecessary shadows. High shutter speeds were used to avoid blurring. These pictures were processed by edge detection in MATLAB to determine the bubble size distribution. The effect of frothers on bubble size is shown at 10 ppm concentration in FIG. 6, which provides the estimated bubble size distribution for each individual frothing agent.

From FIG. 6 it can be noted that for polyglycol type frothing agents, bubble size increases with increasing molecular weight or chain length of the frother. This coincides with other studies indicating a similar trend. It is also noted that MIBC generates larger bubbles than polyglycols at the same frother concentration, which is also shown in FIG. 6. The frothing agent DF200 gave a narrow size distribution with smaller bubble size, making it ideal for this process. Although frothing agents DF250 and AF68 have the same size range, frothing agent AF68 has a wider bubble size distribution, which makes its initial CO₂ absorption efficiency slightly less than frothing agent DF250, which is also observed in FIG. 5. Frothing agents DF400 and AF70 gave similar bubble size distributions but with larger bubble size, making the effective mass transfer area less, which is reflected in the percentage of CO₂ absorption efficiency in FIG. 5.

### Effect of Frother Concentration on CO₂ Absorption

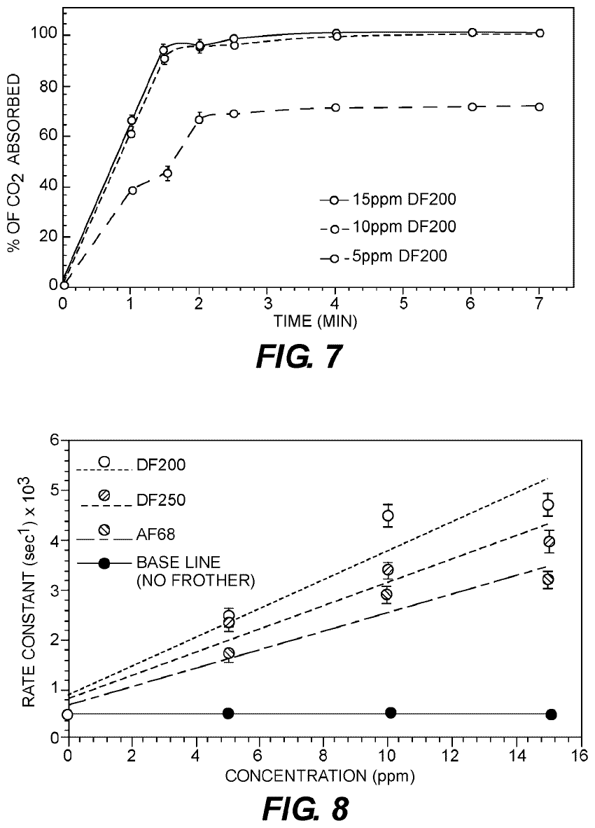

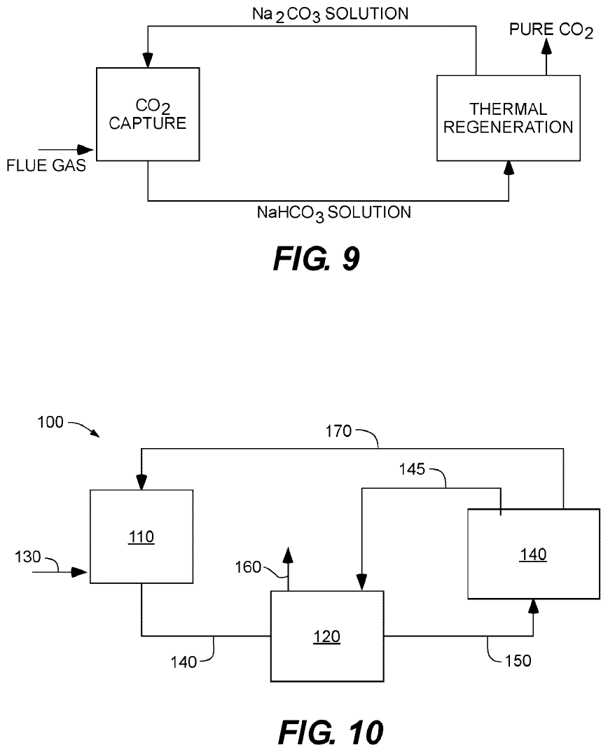

Various concentrations of frothing agents DF200, DF250 and AF68 were tested to study the effect of frother concentration on absorption performance. Since frothing agent DF200 gave the best absorption performance, three different concentrations (5 ppm, 10 ppm and 15 ppm) of frothing agent DF200 is shown in FIG. 7. The frother concentration of 10 ppm was determined to be the optimum dosage for frothing agent DF200. The data suggests that a solution enhanced with less frothing agent requires slightly more time for the amount of CO₂ in the exhaust stream to reach its minimum. This is believed to be due to the amount of time required for stable bubble formation. The froth forms very readily when larger concentrations of frothers are used in the scrubbing solution. For very short batch processes, using a higher concentration of frother is advantageous, as it captures slightly more CO₂ during the early stages of the process. During longer absorption periods, or continuous process, the difference in the bubble building period becomes negligible. With the addition of 10 ppm of the frothing agent DF200, the scrubbing efficiency of sodium carbonate slurry reached 99.9% after reaching steady state, and any further addition of the frother resulted in only very minimal improvements.

Foaming is usually observed when higher concentrations (more than 15 ppm) of surfactants are used. Thus, the tests were restricted to a concentration range of 5-20 ppm of the frothing agent. With lower concentrations, the frothers are aimed towards generating uniform bubble characteristics rather than stable froth/foam formation. Using too high of a concentration of the frothing agent may cause adverse effects such as foaming, where the gas gets completely trapped in the bubble swarm. Table 4 compares the CO₂ capture efficiency of different reagents with frother enhanced sodium carbonate solution.

TABLE 4
Effect of solvent type on CO₂ capture efficiency.

| | CO₂ capture |
|---|---|
| Absorbent | efficiency (%) |
| 0.2M Na₂CO₃ | 55.60 |
| 0.2M NaOH | 97.01 |
| 0.2M MEA | 97.12 |
| 0.2M Na₂CO₃ + 10 ppm DF200 frothing agent | 99.90 |

### Absorption Kinetics

The rate of the absorption reaction was estimated by calculating the slope between the number of moles of CO₂ absorbed versus time. The number of moles absorbed was calculated by performing trapezoidal integration on the graph generated by the data logger on the gas analyzer. The rate constant was estimated from Equation (8), assuming first order kinetics based on the work of Sharma and Danckwerts. The rate constant is directly correlated to the rate of absorption. From FIG. 8, it is evident that rate of absorption is highest with the frothing agent DF200, closely followed by frothing agents DF250 and AF68, which are all polyglycols. Compared to the baseline, these three frothers increased the absorption rate of Na₂CO₃ solution significantly. Though sodium carbonate solution by itself has a lower absorption efficiency than NaOH or MEA, the addition of a frothing agent increased the absorption efficiency of Na₂CO₃ above NaOH and MEA. Additionally, the frother is expected to have no impact on the energy cost of regeneration.

### Reagent Regeneration

Reagent regeneration energy was estimated from the heat duty (2.65 kwh) from heat recycle loop shown in FIG. 3. With 1.13 mol per minute of CO₂ absorbed, heat requirement for the frother-enhanced Na₂CO₃ was around 3.18 MJ/KgCO₂. The frothers had no impact on the energy of the reagent regeneration, perhaps because of their very low concentrations. The typical regeneration energies for MEA-based CO₂ capture have been reported to be around 3.9-4.3 MJ/KgCO₂. The energy consumption utilizing a frother-modified dilute sodium carbonate solution based system was much lower than the MEA based system. Thus, it is believed that using a frother-enhanced dilute sodium carbonate solution based system will reduce the reagent cost and also other operating costs for post-combustion CO₂ capture. The concentration of 10 ppm of the frothing agent DF200 gave the best results among other frothing agents. Originally, increasing frother concentrations increased the absorption rate as seen in FIGS. 7 and 8, but over longer trials and after reaching steady state, this gap was negligible. Owing to the very low concentration of the frothing agents used, the solvent regeneration energy remained essentially the same as a sodium carbonate solution without any frothing agent.

While the frothing agents were observed to degrade at various points throughout the system after 3-4 cycles, a fresh batch of the frothing agent was added after every 3 cycles. It is contemplated that the sodium carbonate solution can be periodically dosed with a frothing agent to address the frothing agent degradation. In some other preferred aspects, a small dose of the frothing agent may be continually added to the sodium carbonate solution to maintain approximately the desired concentration of the frother-modified sodium carbonate solution based system.

The frothing agents did not enter the CO₂ rich stream when the sodium carbonate and CO₂ was regenerated from the sodium bicarbonate, which is believed to be due to their high decomposition temperature being between about 200° C. and about 250° C. compared to the desorption temperature of the system being between about 80° C. and about 110° C., more preferably between about 85° C. and about 105° C., and even more preferably between about 95° C. and about 100° C.

Before any discharging of process water, the organic nature of the frothing agents allows their easy removal with activated carbon, because of their hydrophobicity. A complete guide on low cost flotation frothers treatment methods was reviewed by Li et al. in 2019. Considering the recyclability and based on costs from Table 1 of the Li et al. article, the reagent cost for CO₂ capture could be reduced by at least 50%, in some aspects at least 55%, in some aspects at least 60%, in some aspects at least 65%, in some aspects at least 70%, in some aspects at least 75%, and in some other aspects up to about 78%, by switching to a frother-enhanced sodium carbonate system.

Although amines and NaOH have a very high CO₂ capture efficiency, there are some drawbacks associated with both the reagents including equipment corrosion, solvent degradation, high cost, and so on. The frother-enhanced sodium carbonate system of the present invention enhances the absorption performance of already existing low-cost sodium carbonate solutions while providing an environmentally friendly and non-corrosive solution.

II. Simultaneous Removal of CO₂, NOₓ and SOₓ Using Single Stage Absorption Column

The present inventors have simultaneously captured CO₂, NOₓ and SOₓ from flue gas with a single wet scrubbing column. The absorption of all three gases was achieved using a scrubbing solution comprising sodium carbonate solution promoted with one or more oxidizers in a single stage absorption column.

In the present disclosure, the oxidant is chosen from the oxidizers consisting of H₂O₂, NaOCl, NaOCl₂, and NaClO₃. In some preferred aspects, the oxidant is H₂O₂, NaOCl, or a mixture thereof. H₂O₂ is a very strong oxidizer and has high nucleophilic reactivity for carbonyl carbon. While H₂O₂ is a very strong oxidizing agent, it is also more expensive than other oxidizers. Thus, substituting at least a portion of the H₂O₂ with NaOCl can reduce the reagent cost. In some aspects, NaOCl is preferred over NaClO₂ and NaClO₃ based on the previous observations that ClO— acts as a better nucleophile compared to the other two species.

The present inventors have examined the absorption efficiency of sodium carbonate solution promoted with hydrogen peroxide (H₂O₂) and sodium hypochlorite (NaOCl) on NO, CO₂ and SO₂ under alkaline conditions, including the pH range from about 10.6 to about 11.8. This process with respect to NO is similar to selective non-catalytic reduction (SNCR) at ambient temperature. While sodium carbonate displays slower absorption kinetics for CO₂ absorption compared to traditional amines, adding these rate promoters can enhance the absorption kinetics greatly making its absorption performance surpass that of amines. SO₂ is instantaneously absorbed into aqueous sodium carbonate solutions. One of the unique aspects of the present invention is that the inventors have surprisingly discovered successful absorption of NO, CO₂ and SO₂ gases with a single stage absorption column of sodium carbonate supported with H₂O₂/NaOCl.

The absorption kinetics of both H₂O₂ and NaOCl with all three gases were individually studied. The absorption characteristics of a combined gas system for NO, CO₂ and SO₂ gases and how the absorption kinetics of each individual gas is affected by the rate promoter are also disclosed further herein.

Without wishing to be bound by theory, the reason for adding rate promoters for post combustion CO₂ capture is that sodium carbonate has slower kinetics compared to amines and other alkali absorbents like NaOH. There are several rate promoters that will increase the kinetics as well as aid in using low concentrations of the reagents by achieving high mass transfer ratio in less time.

### CO₂ Absorption in Aqueous Solution

When CO₂ is introduced in aqueous solution, the first step is hydration where gas phase CO₂ is transferred to liquid phase CO₂ then it forms carbonic acid, which reacts with sodium carbonate to form sodium bicarbonate. The reaction between sodium carbonate and CO₂ are shown in Eqs. (6)-(9) below:

Na₂CO₃₍ₛ₎+H₂O₍ₗ₎+CO₂(g)→2NaHCO_{3(aq)} (6)

Eq. (16) represents the overall reaction between aqueous sodium carbonate and CO₂ forming sodium bicarbonate, with the following reaction Intermediates.

CO_{2(g)}═CO₂₍ₗ₎ (7)

CO₂₍ₗ₎+H₂O═H⁺+HCO⁻₃ (8)

HCO⁻₃═H⁺+CO₃²⁻ (9)

Step (18) is the slowest and rate determining step, so adding a rate promoter would enhance the reaction kinetics and improve the absorption efficiency of carbonate solution. Also, the rate of reaction of CO₂ in alkaline solutions follow first order kinetics. Enhancing the reaction kinetics for CO₂ absorption in carbonate solution can be done with the help of several rate promoters like vanadate, hypochlorite, piperazine, etc. Boric acid, arsenous acid and MEA are among other homogeneous rate enhancing reagents previously explored. Arsenous acid gave very good performance for increasing absorption kinetics of CO₂ hydration, but due to toxic and carcinogenic effects of arsenite it is no longer explored as a rate promoter for CO₂ capture. Other reagents like piperazine and boric acid do not have oxidative properties like hypochlorite to enhance NO absorption.

### NO Absorption in Aqueous Solution

NO has very low solubility in water (0.0056 mg/100 mL at 293K). While NO₂ hydrolyses readily in water, if NO can be oxidized to NO₂ then it can be easily absorbed into aqueous solutions. There are several oxidizing agents like H₂O₂, NaClO, NaClO₂, KMnO₄ etc. Other absorbents include Na₂SO₃, FeSO₄, EDTA and urea. Most of the reactions follow first order kinetics. Many of these reagents have disadvantages pertaining to mixed gas system. For example, the use of potassium permanganate has been known to produce brown precipitates, due to the formation of manganese dioxide. These precipitates clog the packing material in the scrubbing column, and also causes problems in the pumping system. Urea has dormant activity for CO₂ and SO₂. NO absorption in aqueous solutions after being oxidized to NO₂ is shown below and the overall reaction of NO and H₂O₂ in the aqueous phase is as follows in Equations 10 and 11:

NO+H₂O₂→NO₂+H₂O (10)

2NO₂+H₂O→HNO₃+HNO₂ (11)

The Reactions Scheme with NaOCl is as Follows in Equations 12-14:

NO+NaOCl→NO₂+NaCl (12)

3NO₂+H₂O→2HNO₃+NO (13)

2NO₂+H₂O→HNO₃+HNO (14)

### SO₂ Absorption in Aqueous Solution:

Although different methods have been proposed over the years, wet scrubbing process is the commonly used process for removing SO₂ from flue gas. The following reaction pathways 15-18 should be considered when sulfur dioxide is introduced into aqueous solutions of NaHCO₃/Na₂CO₃:

SO₂+H₂O═H++HSO⁻₃ (15)

HSO⁻₃═H⁺+SO²⁻₃ (16)

H₂O═H⁺+OH⁻ (17)

HCO⁻₃═H⁺+CO²⁻₃ (18)

Reaction (15) has very fast kinetics, with a forward rate constant reported as 3.40×10⁶ sec⁻¹. Reactions (16) and (17) can be regarded as almost instantaneous, since they are based on simple transfer of H⁺. The mass transfer coefficient of SO₂ in aqueous solutions is correlated to temperature and with increase in temperature it increases, at the operating temperature of around 318K the mass transfer coefficient of SO₂ in aqueous solution is reported to be two times higher than at 293K. Owing to high mass transfer coefficient and instantaneous reactions, SO₂ can be absorbed readily into sodium carbonate solution with or without the presence of rate enhancing reagents.

### Kinetic Measurements

Dankwerts surface renewal model is the widely accepted kinetic model for the absorption of gases in liquid solutions. Based on the Danckwerts film renewal model the rate of absorption of NO is given by Equation 19:

$${N}_{N O} = kg / RT ( {p}_{NO} - {p}_{NOi} ) \qquad ( 19 )$$

where R is universal gas constant, kg (m/sec) is gas phase mass transfer coefficient, T is the temperature and ^{p}_{NO} is partial pressure of NO. ^{p}_{NOi} is the interfacial pressure of NO in the aqueous solution that can be obtained by Henry's law given by Equation 20:

$${p}_{{NO}_{i}} = {H}_{NO} {𝒞}_{NO} \qquad ( 20 )$$

where, H_{NO} (Pa·m³/mol) is Henry's law constant, C_{NO} (mol/m³) is the concentration of NO at the gas-NaClO/Na₂CO₃ solution interface, and is directly associated with the solution's ionic strength. This relationship is shown in the following expression of Equation 21:

$$log ( {𝒞}_{N O} / 𝒞 NOW ) = - ( {k}_{NaClO} {I}_{NaClO} + {k}_{{OH}^{-}} {I}_{{OH}^{-}} ) \qquad ( 21 )$$

where k_{NaClO} and k_{OH}—are the salting-out parameters of NaClO and OH⁻, respectively, I (mol/L) is the ionic strength of the solution, and C_{NOW} (mol/m³) is the interfacial concentration of NO at the gas-water interface. The salting out parameters of an electrolyte solution can be obtained by adding their anion, cation and gas contribution numbers respectively, as shown in Equation 22 below.

$$k = {x}_{a} + {x}_{c} + {x}_{g} \qquad ( 22 )$$

where xₐ is contribution by anions, xc is contribution by cations and x_{g} by gas, respectively in mol/L. One of ordinary skill in the art will appreciate that the individual x values can be identified from previous literature. But, x_{ClO-} is not mentioned in the literature so it is presumed that the role of hypochlorite ion is the same as that of the reported chlorite i.e. x_{ClO-}=0.3497. The rate at which CO₂ is absorbed into carbonate solutions can be described as follows in Equation 23:

$${R}_{CO 2} = d c / dt = {k}_{L} a ( {c}^{*} - c ) = k [ {CO}_{2} ] \qquad ( 23 )$$

where k_{L} is mass transfer coefficient, a is gas-liquid interfacial area, c* is CO₂ concentration at saturation i.e. the solubility of CO₂, c is bulk concentration of CO₂ dissolved, and k is the rate constant assuming first order kinetics. The percentage concentration of gases going in and out of the scrubbing column is continuously monitored by the gas analyzer. The absorption efficiency (AE, %) or percentage of absorbance (PA, %) for each gas (CO₂, NO and SO₂) is calculated individually by the following Equation 24:

$$AE = PA = ( {Y}_{i n} - {Y}_{out} ) / {Y}_{in} \times 100 \% \qquad ( 24 )$$

where Yin is number of moles of the gas going into the scrubbing column and Yout is number of moles of the gas coming out of the scrubbing column.

In the present invention, CO₂, NOₓ and SOₓ can be simultaneously captured from flue gas with a scrubbing unit comprising a scrubbing solution. The absorption of CO₂, NOₓ and SOₓ from glue gas can be achieved using a single stage absorption unit comprising a scrubbing solution, the scrubbing solution preferably comprising a sodium carbonate solution promoted with at least one oxidizer.

The single stage absorption unit preferably comprises a single wet scrubbing column, more preferably a counter-current absorption column.

In some preferred aspects, the scrubbing solution comprises a sodium carbonate solution having a concentration greater than 0.1 mol/L up to 1 mol/L, preferably between about 0.1 mol/L and about 0.8 mol/L, and more preferably between about 0.1 mol/L and about 0.4 mol/L. In some preferred aspects, an optimal concentration of the sodium carbonate solution is between about 0.15 mol/L and about 0.25 mol/L.

The absorption efficiency of the sodium carbonate solution in simultaneously capturing CO₂, NOₓ and SOₓ can be increased by the scrubbing solution also comprising at least one oxidizer. In some aspects, the at least one oxidizer is chosen from the group consisting of H₂O₂, NaOCl, NaOCl₂, NaClO₃, and mixtures thereof. In some preferred aspects, the at least one oxidizer is H₂O₂, NaOCl, or a mixture thereof. In some preferred aspects, the absorption efficiency of the sodium carbonate solution is increased by the addition of H₂O₂ as the oxidizer. In some other preferred aspects, the absorption efficiency of the sodium carbonate solution is increased by the addition of NaOCl as the oxidizer.

In some preferred aspects, the at least one oxidizer is present in the scrubbing solution in an amount between about 100 μL/L and about 1500 μL/L, in some aspects preferably between about 500 μL/L and about 1000 μL/L, and in some aspects more preferably between about 650 μL/L and about 8500 ML/L.

In some preferred aspects, the at least one oxidizer comprises H₂O₂, NaOCl, or a mixture thereof, which is present in the scrubbing solution in an amount between about 100 μL/L and about 1500 μL/L, in some aspects preferably between about 500 μL/L and about 1000 μL/L, and in some aspects more preferably between about 650 μL/L and about 8500 μL/L.

The sodium carbonate solution preferably has a pH in the range between about 8 and about 13, in some aspects between about 9 and about 12.5, in some aspects between about 10 and about 12.2, and in some preferred aspects between about 11 and about 12.

According to some aspects, the absorbance of CO₂ is at least 95%, in some aspects at least 97%, in some aspects at least 99%, in some aspects at least 99.1%, in some aspects at least 99.2%, in some aspects at least 99.3%, in some aspects at least 99.4%, in some aspects at least 99.5%, in some aspects at least 99.6%, in some aspects at least 99.7%, in some aspects at least 99.8%, in some aspects at least 99.9%, and in some aspects 100%, of the flue gas entering the scrubbing unit.

In some aspects, the absorbance of NO is at least 25%, in some aspects at least 26%, in some aspects at least 27%, in some aspects at least 28%, in some aspects at least 29%, and in some aspects at least 30%, of the flue gas entering the scrubbing unit.

In some aspects, the absorbance of SO₂ is at least 90%, in some aspects at least 91%, in some aspects at least 92%, in some aspects at least 93%, in some aspects at least 94%, and in some aspects at least 95%, of the flue gas entering the scrubbing unit.

In some aspects, the absorbance of the flue gas entering the scrubbing unit is at least about 95% for CO₂, at least about 25% for NO, and at least about 90% for SO₂, in some aspects at least about 97% for CO₂, at least about 26% for NO, and at least about 91% for SO₂, in some aspects at least about 98% for CO₂, at least about 27% for NO, and at least about 92% for SO₂, in some aspects at least about 99% for CO₂, at least about 28% for NO, and at least about 92% for SO₂, in some aspects at least about 99.2% for CO₂, at least about 28.5% for NO, and at least about 92.5% for SO₂, in some aspects at least about 99.5% for CO₂, at least about 29% for NO, and at least about 93% for SO₂, and in some preferred aspects at least about 99.7% for CO₂, at least about 29.5% for NO, and at least about 94.5% for SO₂.

In some aspects, the absorbance of the flue gas entering the scrubbing unit is up to about 100% for CO₂, up to about 50% for NO, and up to about 99% for SO₂, in some aspects up to about 100% for CO₂, up to about 45% for NO, and up to about 98% for SO₂, in some aspects up to about 100% for CO₂, up to about 40% for NO, and up to about 97% for SO₂, and in some other aspects up to about 100% for CO₂, up to about 35% for NO, and up to about 97.5% for SO₂,

In some aspects, an inlet temperature of flue gas comprising CO₂, NOₓ and SOₓ entering the scrubbing unit, preferably the single stage absorption unit, is between about 32° C. and about 52° C., preferably between about 34° C. and about 50° C., more preferably between about 36° C. and about 48° C.

In some aspects, a ratio of the scrubbing solution to flue gas in the single stage absorption unit is between about 2 to about 5, preferably between about 3 and about 4.8, more preferably between about 4 and about 4.6.

In some aspects, a scrubbing solution flow rate is between about 1 gallon/minute and about 5 gallons/minute, preferably between about 1.25 gallons/minute and about 4 gallons/minute, more preferably between about 1.5 gallons/minute and about 2.5 gallons/minute.

The single stage absorption unit preferably also comprises a packing material. In some aspects, the packing material comprises pall rings. The pall rings can comprise polypropylene, polyvinylidene fluoride (PVDF), high-density polyethylene (HDPE), glass filled polypropylene, polyvinyl chloride (PVC), and combinations thereof. In some preferred aspects, the packing material comprises polypropylene pall rings.

The scrubbing solution is preferably essentially devoid of other rate-enhancing agents, including piperazine (PZ), monoethanolamine (MEA), boric acid, carbonic anhydrase (CA) and polyglycol ethers.

In some aspects, the present invention comprises a scrubber system for the simultaneous removal of CO₂, NOₓ and SOₓ from flue gas. In some preferred aspects, the scrubber system comprises a single stage absorption column and a scrubbing solution within the single stage absorption column, wherein the scrubbing solution comprises a sodium carbonate solution promoted with at least one oxidizer.

In some aspects, the scrubber system comprises a second scrubbing column in series after the single stage absorption column, wherein the second scrubbing column is configured for removal of a remaining amount of NOₓ from the single stage absorption column.

In some aspects, the scrubber system comprises a primer scrubbing column in series prior to the single stage absorption column, wherein the primer scrubbing column is configured for removal of an initial amount of NOₓ from the flue gas.

In some aspects, the second scrubbing column and/or the primer scrubbing column comprises a selective catalytic reduction scrubbing column.

In some aspects, the present invention comprises a method for the simultaneous removal of CO₂, NOₓ and SOₓ from flue gas using a single stage absorption unit having a scrubbing solution comprising sodium carbonate solution and at least one oxidizer. The method includes providing a scrubbing unit comprising a single stage absorption unit having a scrubbing solution therein, the scrubbing solution comprising a sodium carbonate solution promoted with at least one oxidizer. Flowing flue gas comprising CO₂, NOₓ and SOₓ through the scrubbing unit. In some preferred aspects, the scrubbing solution has a flow in an opposite direction of the flue gas, such that the scrubbing unit is used as a counter-current absorption unit. In some preferred aspects, the scrubbing solution is continuously fed into the single stage absorption unit at an opposite end as the flue gas, such that clean gas exits the same end as the scrubbing solution being entered into the single stage absorption unit.

In some preferred aspects, scrubbing solution is provided at an alkaline pH. The sodium carbonate solution preferably has a pH in the range between about 8 and about 13, in some aspects between about 9 and about 12.5, in some aspects between about 10 and about 12.2, and in some preferred aspects between about 11 and about 12.

An inlet temperature of flue gas comprising CO₂, NOₓ and SOₓ entering the scrubbing unit, preferably the single stage absorption unit, is between about 32° C. and about 52° C., preferably between about 34° C. and about 50° C., more preferably between about 36° C. and about 48° C.

A ratio of the scrubbing solution to flue gas in the single stage absorption unit is between about 2 to about 5, preferably between about 3 and about 4.8, more preferably between about 4 and about 4.6.

A scrubbing solution flow rate is between about 1 gallon/minute and about 5 gallons/minute, preferably between about 1.25 gallons/minute and about 4 gallons/minute, more preferably between about 1.5 gallons/minute and about 2.5 gallons/minute.

The single stage absorption unit preferably also comprises a packing material. In some aspects, the packing material comprises pall rings. The pall rings can comprise polypropylene, polyvinylidene fluoride (PVDF), high-density polyethylene (HDPE), glass filled polypropylene, polyvinyl chloride (PVC), and combinations thereof. In some preferred aspects, the packing material comprises polypropylene pall rings.

### Example

### Materials and Methods

### Column Properties

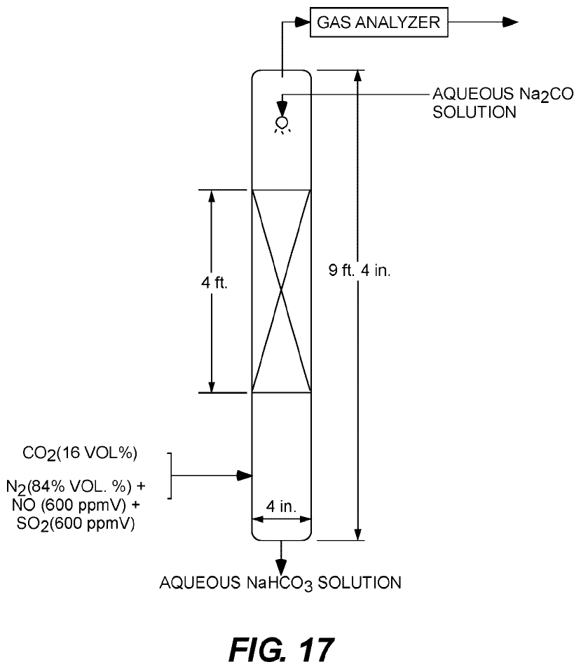

A CO₂ capture column has been designed and built as shown in FIG. 17. The packing material used to fill the scrubber column is polypropylene pall rings 0.5 inch×0.5 inch. The height of a packed bed scrubbing column (Z) is calculated using the contact tower design equation (Equation 25 below). Gs represents molar flow of solute-free gas per cross-sectional area of the column. a is the interfacial area available for mass transport. Ky accounts for overall gas phase mass transfer coefficient. Y is the fraction of moles of gas phase solute per moles of solute-free gas, and Y* denotes the gas phase mole fraction in equilibrium with the liquid phase. The denominator of the integral represents the driving force for mass transfer and is integrated over the condition of the gas phase from the top to the bottom of the column.

$$Z = \frac{{G}_{s}}{{K}_{y} \times a} {{\int }^{{Y}_{1}}}_{{Y}_{2}} \frac{dY}{Y - {Y}^{*}} \qquad ( 25 )$$

Given that the interfacial area a is in the denominator of the design equation, it is advantageous to have a large amount of interfacial area within the scrubbing column. This is the reason most scrubbing columns are filled with packing.

### EXPERIMENTAL

The pilot scale scrubbing column shown in FIG. 17 was used to conduct experiments on absorbance of CO₂, NO and SO₂ with sodium carbonate solution in the presence of oxidizer. The top portion of the capture column (7 ft) is made of transparent poly acrylic plastic, and the bottom portion is made of steel to ensure robustness. The packed-bed absorption column (Packing: Polypropylene pall rings 0.5 inch×0.5 inch) in FIG. 17 is used as a counter-current absorption column, where flue gas enters from the bottom of the column, then the gas flows up through the packed bed where it contacts the scrubbing liquid. The scrubbing liquid removes the contaminant and exits out the bottom. Clean gas then exits out from top of the column. In order to simulate the flue gas, a gaseous mixture containing 16 vol. % CO₂, 600 ppmV NO, 600 ppmV SO₂ and remainder nitrogen was continuously fed into the bottom of the scrubbing column with the help of a gas diffuser.

For the absorption experiments, Na₂CO₃ (99.8% pure) was obtained from Genesis Alkali, H₂O₂ and NaOCl (reagent grade) were obtained from Sigma-Aldrich. All gas cylinders were obtained from Air-products. Gas flow rate was maintained at 21 L/min. Gas flow rates were measured with gas flow meters (Model 7520, OMEGA, USA) equipped with gas controllers (Model 316, McMaster-Carr, USA). Separate flow meters were installed for the mixed gases to measure the volumetric flow and to control the percentage of CO₂ in the gas stream. Stainless steel is suggested for the column and piping to avoid any equipment corrosion due to caustic pH.

The composition of gases exiting out from the top of the column is measured with a Nova Multi-Gas Analyzer fitted with nondispersive infrared (NDIR) and electrochemical sensors, calibrated with CO₂/NO/SO₂/N₂ reference gases. A range of concentrations for the oxidizer (H₂O₂/NaOCl) starting from 500 to 1500 μL/L were tested. The pH measurements were taken at regular intervals with Oakton handheld pH meter. The percentage of absorbance data was continuously recorded by the data logger connected to the gas analyzer. After each experiment the data logger was connected to the computer and the graph generated from it was integrated to calculate the total moles of CO₂ absorbed per minute for measuring the kinetic data. The accuracy of the data was ensured by repeating these experiments in triplicates. The operating conditions of the column shown in FIG. 17 were: liquid/gas-ratio 4.3, scrubbing solution flowrate 2 gallons/min, gas inlet temperature 313K, scrubbing solution inlet temperature 318K, gas composition 16 vol. % CO₂, 600 ppm V NO, 600 ppm V SO₂, and rest N₂.

### Results and Discussion

Along with replacing three stage flue gas capture with single stage, it is also an aim of the present invention at reducing the reagent costs by switching from amines to dilute sodium carbonate solution enhanced with rate promoters. CO₂ capture with dilute sodium carbonate solution was first disclosed in U.S. Pat. No. 7,919,064. Later there were several improvements made for this process, most recently in Barzagli et al. (2017) having tested dilute sodium carbonate solution for CO₂ capture and were able to achieve 80% CO₂ absorption efficiency. The present inventors have tested various concentrations of sodium carbonate solution ranging from 0.1 to 0.4 mol/L with the addition of H₂O₂/NaOCl ranging from 500 to 1000 μL/L. Starting with a 50-gallon solution, the scrubbing solution was recycled through the scrubber for a total duration of 87 min before it is completely loaded with bicarbonate. After performing several experiments, the optimum concentration was noted to be 0.2 mol/L Na₂CO₃ solution+750 μL/L H₂O₂, achieving 99.7% absorbance for CO₂, 31% for NO and 97% for SO₂ respectively. The experimental uncertainty is calculated and the error bars are plotted within the 95% confidence interval. These results, along with reaction kinetics are discussed in detail in further sections.

### CO₂ Absorption

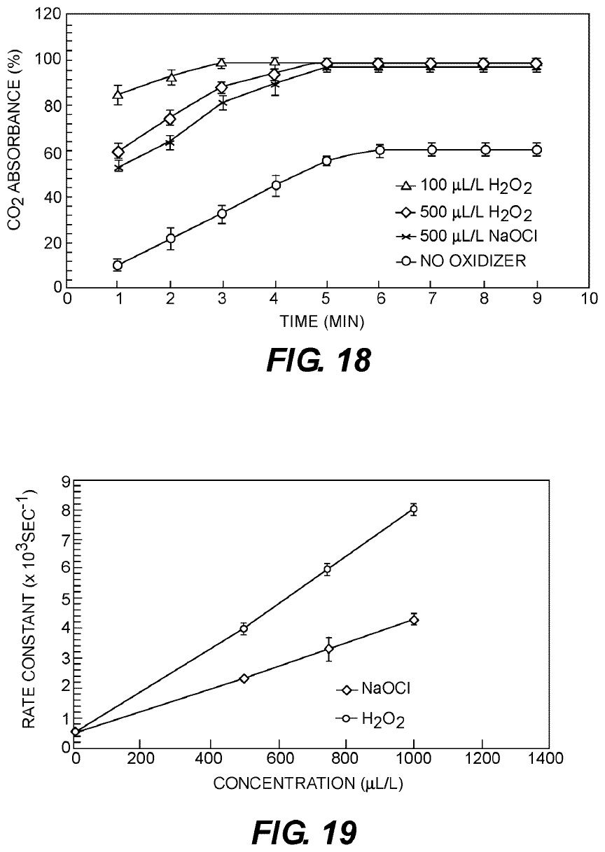

Curves in FIG. 18 show the absorbance of CO₂ in 0.2 mol/L Na₂CO₃ solution enhanced with H₂O₂/NaOCl. The percentage of absorbance (%) reached 80% in the first 1 min with the addition of H₂O₂/NaOCl and finally reaching 99.7% in 5 min after reaching steady state. The absorbance with Na₂CO₃ solution alone is only 61%, but after the addition of oxidizer the absorbance increased to 100%. The rate of absorption increased with increasing H₂O₂ and NaOCl concentrations. Initially with increase in concentration of the oxidizer from 500 to 1000 μL/L showed increased kinetics of CO₂ absorption, but after reaching steady state in 5 min, 750 and 1000 μL/L oxidizer gave almost similar absorption efficiency, with 1000 μL/L concentration showing 0.2% higher absorption than 750 μL/L. While performing the experiment, the inventors observed effervescence in the liquid solution, though barely visible. In addition to potential chemical kinetic effects, the effervescence is believed to have led to additional bubble formation, increasing the mass transfer area of contact between the gas and the liquid. In Equation 33 increasing the interfacial area (a) will increase the absorption rate.

### CO₂ Absorption Kinetics

The reaction intermediates for CO₂ absorption into sodium carbonate solution are given below (Steps (2)-(4)). Step (3) is the rate determining step, since the rest of the reactions are almost instantaneous.

CO_{2(g)}═CO₂₍ₗ₎ (Step 2)

CO₂₍ₗ₎+H₂O═H⁺+HCO⁻₃ (Step 3)

HCO₋₃═H++CO²₃ (Step 4)

Adding a small amount of rate promoters can enhance the CO₂ absorption capacity of carbonate solutions significantly at lower temperatures. Since CO₂ is a Lewis acid, Lewis bases with O⁻ or OH groups can act as rate promoters. The enhanced CO₂ absorption rate in FIG. 18 can be attributed to the rate enhancing activity of H₂O₂/NaOCl on the equilibrium rate determining reaction (Step 3). The time required to establish equilibrium was reduced after the addition of H₂O₂/NaOCl. Whether its organic or inorganic additive, both follow a mechanism suggested by the Astarita equation as shown below in Equations 26 and 27:

CO₂+Promoter→Intermediate (26)

Intermediate+OH₋→HCO₃₋+Promoter (27)

For the homogeneous activity with H₂O₂ and NaOCl, carbonyl carbon acts as the substrate. This reaction scheme can be seen below. In case of homogeneous catalysis in the presence of H₂O₂/NaOCl, the step of equation (27) follows the step of equation (26) immediately. In a broader view these additives do not undergo any major chemical transformation, but rather increase the overall mass transfer phenomenon. Reaction mechanisms can be seen in Schemes 1 and 2 based on the alpha effect theory proposed by Edwards and Pearson (1962).

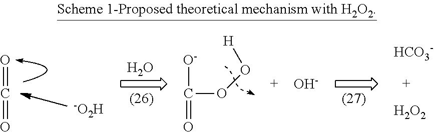

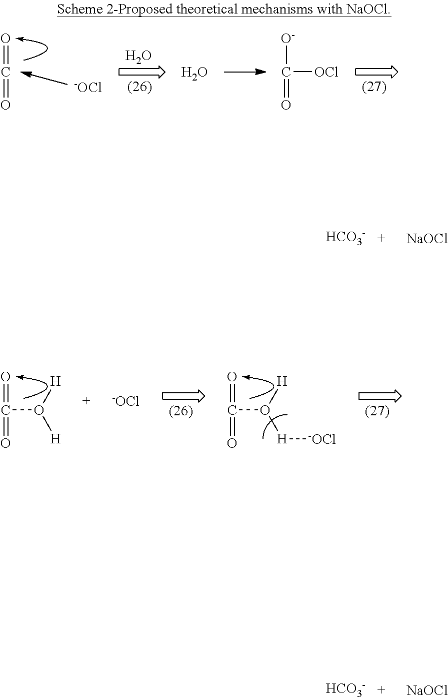

Rate of reaction was estimated by calculating the slope of number of moles of CO₂ absorbed vs time. Number of moles absorbed was calculated by performing trapezoidal integration on the graph generated by the data logger on the gas analyzer. The rate constant shown in FIG. 19 was estimated from the rate of reaction in Eq. (23) assuming first order kinetics. The observed rate constant represents that H₂O₂ is a better homogeneous catalyst than NaOCl. In alkaline pH conditions certain nucleophiles like peroxide and hypochlorite react very rapidly. This nucleophilic substitution is described as “Alpha Effect” by Edwards and Pearson (1962). In this scenario, carbonyl carbon acts as the substrate, so under these conditions the rate constants (rate constants of H₂O₂ 2×10₅ mol−1 min−1, rate constants of NaOCl 1.6×10³ mol⁻¹ min⁻¹ (Edwards and Pearson, 1962; Jencks and Carriuolo, 1960) clearly indicate that peroxide has higher absorption kinetics compared to hypochlorite. A similar trend was observed in the case of CO₂ absorption kinetics with H₂O₂ and NaOCl as shown in FIG. 19, which supports the theory. Few researchers have previously tested ClO₂ and ClO₃ as well. Without wishing to be bound by theory, it is believed that the reason that ClO functions as a stronger nucleophile compared to ClO₂ and ClO₃ is because the exchange of electrons on the oxygen atom in ClOn occurs at a faster rate with a lower n value and thus Cl having a lower oxidation state. Overall H₂O₂ gave slightly better kinetics compared to NaOCl as shown in FIG. 19.

Although the present inventors have not performed liquid analysis, depending on the molar ratio of CO₂ converted and sodium carbonate used (0.2 mol/L), the fraction of sodium carbonate converted to bicarbonate is only 0.45 without the rate promoter, due to slower absorption kinetics. The conversion increased to 82% after the addition of the rate promoter, which corresponds to an increase of 45.1%.

### NO Absorption

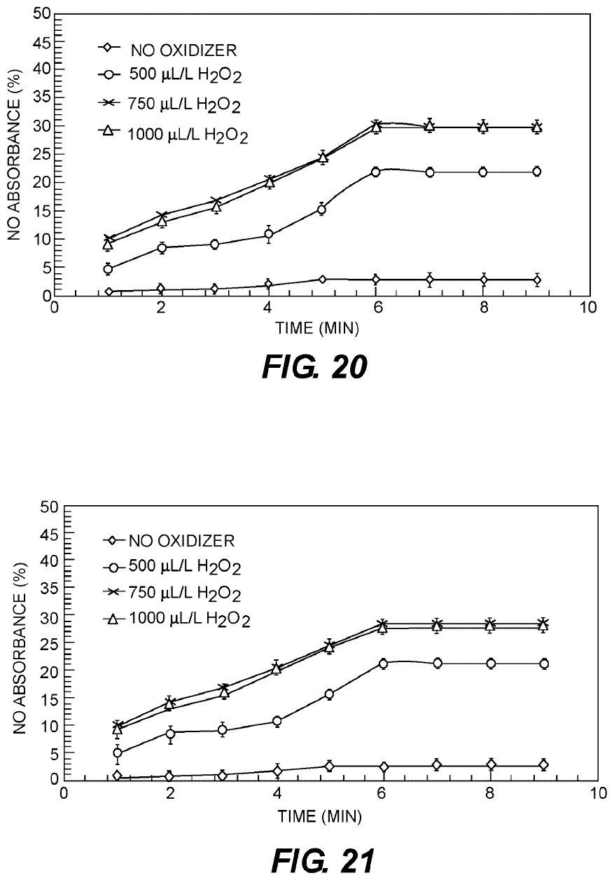

FIGS. 20 and 21 show the percentage absorbance of NO in 0.2 mol/L Na₂CO₃ solution enhanced with 500 to 1500 μL/L H₂O₂ and NaOCl respectively. The percentage of absorbance reached 10% in the first 1 min with the addition of H₂O₂ and finally reaching 31% in 5 min after reaching steady state. The percentage of absorbance reached 9% in the first 1 min with the addition of NaOCl and finally reaching 29% in 5 min after reaching steady state. The NO absorption efficiency increased with increase in oxidizer concentration from 500 to 750 μL/L. The absorbance increased only slightly thereafter and reached an asymptotic maximum at 1000 μL/L concentration. It can be noted that H₂O₂ gave better absorption kinetics than NaOCl, which is discussed in detail below. The absorption performance of both rate promoters is limited at ambient conditions in the absence of a heterogeneous catalyst. The present inventors were able to achieve 30.2% absorbance with 0.2 mol/L Na₂CO₃ solution+1000 μL/L H₂O₂ at pH 11.45 and temperature 318K.

Since the NO oxidation reaction is limited after a certain value at 318K, increasing temperature might increase the absorption performance, but due to other mixed gases and physical limitations of the system, the inventors could not increase the temperature of the absorbent solution. One other possibility is adding a heterogeneous catalyst like platinum to reduce the activation energy and promote the reaction rate at 318K.

Also, pH plays a crucial role in limiting the NO absorption efficiency of the solution. At pH of about 11.5, the reaction tends to limit itself after certain interfacial concentration is reached. As such, the absorbance stopped at 30.2%. In retrospect, NO oxidation continues to increase with increased oxidizer at lower pH values of around 5.

### NO Absorption Kinetics

The absorption rate of NO can be expressed by Equation 28, based on the gas-liquid mass transport theory proposed by Dackwerts and Lannus (1970).

$${R}_{N O} = \sqrt{\frac{2}{m + 1} \times {k}_{m , n} \times {D}_{N O} \times {C}_{N O}^{m + 1} \times {C}_{NaOCl}^{n}} \qquad ( 28 )$$

where R_{NO} is the rate of absorption of NO, k_{m,n} is the rate constant and D_{NO} is the diffusion coefficient of NO in water, which can be considered as 2.076×10⁻⁹ m²/sec at 318K. C_{NO} is the interfacial concentration of NO, which can be obtained from Equation 16. Baveja et al. (1979) studied the absorption kinetics of nitric oxide in hydrogen peroxide solution and concluded that first order kinetics followed. The reaction was found to follow first order kinetics with NaOCl as well. So, the values of m, n are considered to be m=1 and n=1. The rate constant was estimated from Arrhenius equation, where the activation energy (Eₐ) and frequency factor (A) are Eₐ=57.3 KJ/mol, A=6.52×10⁹ m³/(mol·sec) and Eₐ=28.15 KJ/mol, A=7.96×10⁸ m³/(mol· sec) for H₂O₂ and NaOCl, respectively (Baveja et al., 1979; Deshwal and Kundu, 2015).

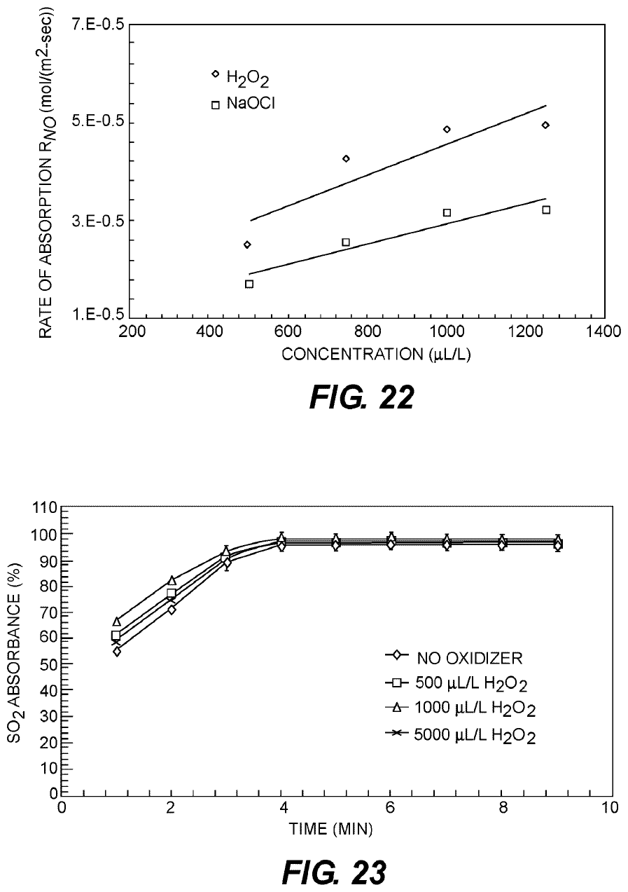

The effect of oxidizer concentration on the rate of absorption of NO at 318K and 0.2 mol/L Na₂CO₃ concentration can be seen in FIG. 22. The rate of absorption of NO initially increases with increasing oxidizer concentration and attains a steady state after 1000 μL/L for both NaOCl and H₂O₂. This can be attributed to the fact that rate constant reaches a limiting value at higher pH levels beyond certain concentration of the rate promoter (Deshwal and Kundu, 2015). At pH>10 the absorption efficiency decreases due to decrease in oxidizing potential of the catalyst. A slowdown of absorption of NO was observed because of the decrease in oxidizing ability of NaOCl at higher pH values. The potential for the half cell reaction of NaOCl in alkaline pH conditions can be seen in Equation 29 below:

ClO⁻+2H⁺+2 e⁻═Cl⁻+H₂O E°=1.48V (29)

where E° is the standard oxidation potential. According to Nernst equation higher H+ concentration implies higher potential (E) and hence higher oxidizing ability. So, at higher pH values the oxidizing power reduces rapidly. Concentration of Na₂CO₃ also has a direct effect on NO absorption efficiency. With increase in Na₂CO₃ concentration from 0.2 to 0.3 mol/L the rate of reaction of NO drastically reduced. Wei et al. (2009) have also observed reduced NO absorption rate with increase in sodium carbonate concentration from 0.01 to 0.05 mol/L with NaClO₂ as the rate promoter. The same applies for other alkali absorbent solutions as well. In case of NaOH as the absorbent solution Sada et al. (1978) have observed an exponential decrease in rate of reaction.

### SO₂ Absorption

Since the reactions (10)-(13) are almost instantaneous, the rate of absorption of SO₂ is very high compared to CO₂ and NO in aqueous medium. FIG. 23 shows the absorbance of SO₂ in 0.2 mol/L Na₂CO₃ solution enhanced with H₂O₂/NaOCl. The absorbance reached 65% in the first 1 min and finally reaching 97% in 5 min after reaching steady state. The oxidizer did not show any major effect. Absorbance reached 95% very fast, hitting a maximum value of 96.2%. The rate promoters show almost negligible/minimal effect on absorption performance of SO₂ in aqueous Na₂CO₃ solution. These rate promoters do increase the absorbance of SO₂ but since it is already readily absorbed, this difference is minute. Presence of NO₂ from NO oxidation have not shown any significant effect on the absorption performance of SO₂.

Effect of Solution pH on Absorption Efficiencies of CO₂, NO and SO₂

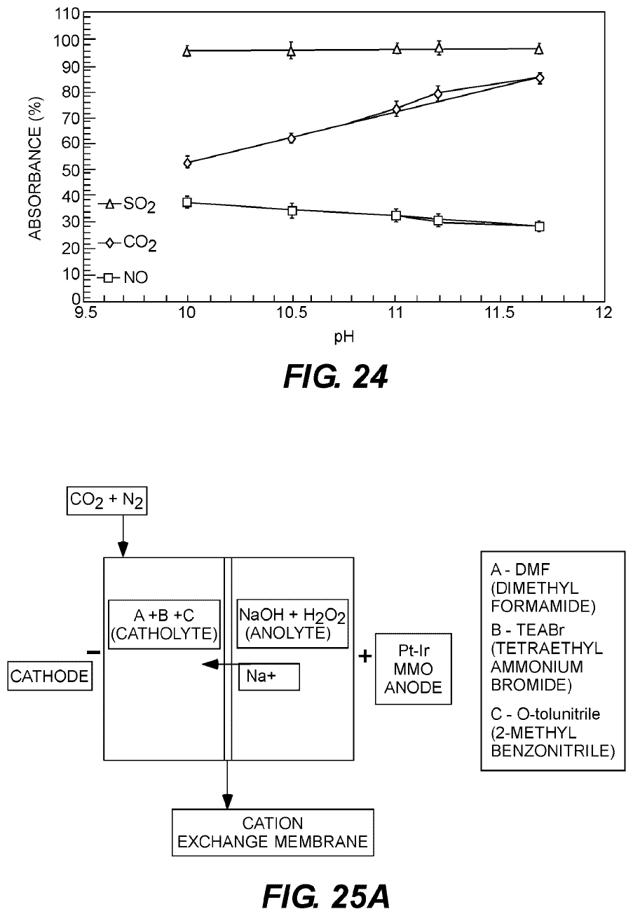

Initial pH of the absorbent solution plays a crucial role in determining the mass transfer rate of gases into liquids. The pH of the solution was varied from 10.62 to 11.73 by changing the Na₂CO₃ concentration. FIG. 24 shows the effect of pH on the absorbance of all the three gases at 750 μL/L H₂O₂ concentration after 5 min of reaching steady state. The absorbance of SO₂ remained mostly unaffected, while that of CO₂ reduces rapidly at lower pH values due to low H+ buffering capacity of the solution. The absorption efficiency for NO increases slightly at lower pH values. As evidenced by previous literature, where they studied NO absorption in acidic pH and observed that with increase in pH, absorption lowered.

Few researchers have previously studied the NO absorption characteristics in acidic pH conditions and observed a decrease in NO oxidation rate with increased pH (Baveja et al., 1979; Deshwal et al., 2008b; Krzyzynska and Hutson, 2012; Myers and Overcamp, 2002). The present inventors have observed quite a similar trend in our study in the pH range of 10 to 12, where the rate of absorption of NO decreased with increased pH, because of the weak ability of H₂O₂/NaOCl to act as an oxidizer in alkaline conditions. Since the primary goal of this unit is to capture CO₂, operating at a pH of 11.6 or higher is ideal.

While the scrubber system of the present invention may not be a substitute for current SCR, it can definitely be used in industrial flue gas treatment with lower concentrations of NOₓ and SOₓ. It is also suggested that wherever the NOₓ percentage is higher, an additional scrubbing column should be included in series combination with the original scrubber, so that whatever NOₓ is left unabsorbed in the first column is absorbed by the second column.

The present invention illustrates that it is possible to capture CO₂, NO and SO₂ with a single scrubbing column. The efficacy of the system is clearly higher with a CO₂ absorption efficiency of 99.7%, compared to previous studies on CO₂ capture using low cost dilute sodium carbonate solution. Absorbance of CO₂ in a sodium carbonate scrubber column increased from 61% to 99.7% after the addition of H₂O₂ or NaOCl. NO was also absorbed, but was limited by the alkaline pH to less than 31% absorbance. Lowering the pH decreased CO₂ absorption while increasing NO absorption. Excessive supply of oxidizer did not improve the absorption efficiency of NO. SO₂ absorption reached 95% almost instantaneously, with or without the addition of oxidizer. H₂O₂ acted as better rate enhancing agent than NaOCl. Enhancing the dilute sodium carbonate solution with H₂O₂ increases its CO₂ absorption performance reducing the need for additional alkaline reagent.

III. Reduced Reagent Regeneration Energy for CO₂ Capture with Bipolar Membrane Electrodialysis

Post combustion CO₂ capture with reagents such as amines, sodium carbonate and sodium hydroxide is the most mature CO₂ capture technology. One of the major challenges facing post combustion CO₂ capture is the high energy requirement for reagent regeneration. Thermal regeneration energy is currently in the range of 3-4 MJ/Kg CO₂ captured.

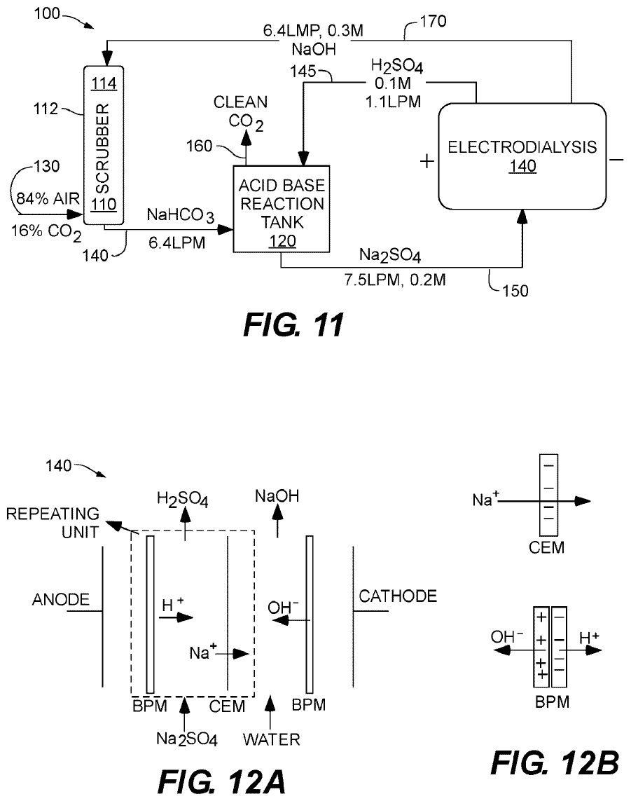

The present inventors were able to significantly reduce reagent regeneration energy by employing electrodialysis with bipolar membrane separation (EDBM), as shown in FIGS. 10 and 11, according to certain embodiments of the present invention.

As shown by the process flow diagram in FIG. 10, CO₂ capture and regeneration system 100 generally comprises CO₂ capture assembly 110 in fluid communication with each of regeneration assembly 120 and EDPM assembly 140, and regeneration assembly 120 being in fluid communication with EDPM assembly 140.

Capture assembly 110 can comprise any apparatus or system for capturing CO₂ from a feedstock. In some preferred aspects as shown by the process flow diagram in FIG. 11, capture assembly 110 comprises a scrubber assembly 110 preferably comprising scrubbing column 112, which contains slurry scrubbing solution 114, and a gaseous feedstock 130 is fed into scrubbing column 112. Slurry scrubbing solution 114 is preferably fed into scrubbing column 112 proximate a slurry solution inlet, which is preferably proximately located a top portion scrubbing column 112. Slurry scrubbing solution 114 can comprise fresh slurry scrubbing solution, regenerated slurry scrubbing solution, or a mixture thereof. Gaseous feedstock 130 preferably comprises a mixture of CO₂ and air, which is preferably fed into scrubbing column 112 proximate a gas inlet, which is preferably proximately located a bottom portion of scrubbing column. Scrubbing column 112 is preferably a packed-bed counter-current absorption column, such that the flow of slurry scrubbing solution 114 is in an opposite direction to the flow of gaseous feedstock 130.

Slurry scrubbing solution 114 and gaseous feedstock 130 are each preferably fed into scrubbing column 112, such that scrubber assembly 110 is capable of providing continuous CO₂ capture. During normal operation, CO₂ is absorbed from gaseous feedstock 130 by slurry scrubbing solution 114 providing resultant product 140, which is preferably a resultant product solution, configured to exit scrubbing column 112 proximate at a resultant product outlet 142, preferably proximately located bottom portion 134 of scrubbing column 112, providing resultant product stream 144. Gaseous feedstock 130 preferably being a flue gas or other carbon dioxide containing gaseous feedstock. A stream of resultant product 140 from scrubbing column 112 can be introduced into regeneration assembly 120. In some preferred aspects, resultant product 140 comprises a sodium bicarbonate solution. In some preferred aspects, a stream of resultant product 140 is continuously introduced into regeneration assembly 120.

Regeneration assembly 120 preferably comprises a reaction tank. Resultant product 140 introduced into regeneration assembly 140 can be reacted with an acid reagent 145 to regenerate CO₂ 160 in a purified form and a resultant salt solution 150. In some preferred aspects, acid reagent 145 comprises sulfuric acid. In some preferred aspects, resultant salt solution 150 preferably comprises a sodium carbonate solution.

Resultant salt solution 150 can be fed to EDPM assembly 140, wherein resultant salt solution 150 can be subjected to electrodialysis with one or more CEM and BPM to separate acid and base as regenerated acid 145 and regenerated base 170, respectively.

Regenerated base 170 can be circulated back to capture assembly 110, which in the instance of a scrubbing assembly to the scrubbing column 112 as scrubbing solution 114 to capture additional CO₂. In instances where regenerated base 170 is circulated back to scrubbing column 112 as slurry scrubbing solution 114, regenerated base 170 can comprise a portion of scrubbing solution 114, such as being mixed with a fresh portion of slurry scrubbing solution 114. In some alternative aspects, regenerated base 170 is continually circulated, such that regenerated base 170 becomes regenerated scrubbing solution that is part and parcel of slurry scrubbing solution 114 once introduced into slurry column 112. In some preferred aspects, regenerated slurry scrubbing solution preferably comprising sodium hydroxide, sodium carbonate, or a mixture thereof. In some other preferred aspects, regenerated slurry scrubbing solution comprises sodium hydroxide.

Regenerated acid 145 can be circulated back to regeneration assembly 120 for additional reaction with resultant product 140 to regenerated CO₂ 160 and resultant salt solution 150.

As provided by the foregoing disclosure of CO₂ capture and regeneration system 100, the process of capture and regenerating CO₂ and the associated reagents can be a continuous process. In certain aspects of the present invention, NaHCO₃ formed in the capture assembly 110 can be subjected to acid regeneration in the presence of an acid to regenerate CO₂ in a purified form and form a resultant salt solution, and then the resultant salt solution can be subjected to EDBM to regenerate an alkali absorbent solution. Employing the acid regeneration and EDBM to the resultant salt solution not only regenerates a high level of CO₂, but the separation of a salt solution into acid and base, such that the starting acid and base reagents are also substantially regenerated. Accordingly, the alkali absorbent solutions that can capture CO₂ from the gaseous feedstock, such as a flue gas, can be recycled and reused, preferably recycled and reused in a continuous manner.

In some aspects, the system and process of the present invention includes capturing CO₂ in a scrubbing column with a scrubbing solution, in some aspects an alkali absorbent solution, in some aspects an alkali metal hydroxide solution, in some preferred aspects sodium hydroxide (NaOH), to form a NaHCO₃ solution, and then regenerating pure CO₂ by an acid regeneration process comprising reacting the NaHCO₃ solution with an acid, in some aspects a mineral acid, in some preferred aspects sulfuric acid (H₂SO₄), to form a resultant salt solution, in some preferred aspects a sodium sulfate (Na₂SO₄) solution.

In some aspects, the resultant salt solution, such as the preferred Na₂SO₄ solution, is subjected to electrodialysis with bipolar membrane (EDBM) for regenerating the starting scrubbing solution, such as the preferred alkali absorbent solution, more preferably the NaOH solution. The EDBM also regenerates the acid that reacts with the NaHCO₃ solution. One unique aspect of acid regeneration is the high recovery of CO₂, which then allows employing the EDBM method to essentially separate the salt solution into acid and base, thus achieving low reagent regeneration energy of CO₂ and the scrubbing solution.

In some aspects, the reagent regeneration of the present invention eliminates the disadvantages of direct electrodialysis of NaHCO₃, such as low current efficiency, low CO₂ recovery (40-60%) and high cell resistance.

Also, switching from toxic reagents like amines to alkali absorbents like sodium carbonate and sodium hydroxide can further save reagent costs. Still further, the cost of reagent regeneration energy utilizing EDBM according to certain embodiments of the present invention is anticipated to even go down further as membrane prices decrease in cost. Solar energy, or other renewable energy sources, can be used for energizing EDBM cell, further minimizing energy costs. These traits not only make the technology of the present invention economically feasible, but also environmentally benign.

The chemical reactions for CO₂ capture with alkali absorbent solutions of NaOH and Na₂CO₃ are shown in Equations (30)-(32):

CO₂(aq)+H₂O(l)→H₂CO₃(aq) (30)

H₂CO₃(aq)+Na₂CO₃(aq)→NaHCO₃(aq) (31)

NaOH(aq)+H₂CO₃(aq)↔NaHCO₃(aq)+H₂O(l) (32)

As provided in FIGS. 2 and 3, the resultant NaHCO₃ solution from these chemical-reaction type capture of CO₂ can be reacted with an acid for CO₂ regeneration and producing a resultant salt solution, and then the resultant salt solution can be subjected to EDBM to regenerate the alkali absorbent solution and the acid.

For example, the reaction in Equation (33) has been found to effectively liberate CO₂ from bicarbonate:

H₂SO₄+2NaHCO₃→Na₂SO₄+2H₂O+2CO₂ (33)

As provided in FIGS. 10 and 11, the Na₂SO₄ salt solution from Equation (33) can be separated back into acid (H₂SO₄) and base (NaOH) by employing the EDBM process. The resultant base solution can be recirculated back for the absorption of CO₂. The present inventors have achieved 100% CO₂ recovery from this regeneration method, with significantly less energy consumption than other regeneration processes. The present invention not only is capable of recovering 100% CO₂ and effectively regenerating the acid and base solutions while utilizing less energy consumption than other regeneration processes, but the present invention eliminates all the disadvantages of direct electrodialysis of NaHCO₃, such as low current efficiency, low CO₂ recovery (40-60%) and high cell resistance.

Without wishing to be bound by theory, the EDBM process uses a bi-polar membrane to specifically catalyze water dissociation to form free protons and hydroxide anions as shown in Equation (34):

H₂O→H⁺+OH⁻ (34)

Then, as shown in FIG. 12A, the EDBM comprises a series of cation exchange membranes (CEM) and bipolar membranes (BPM) proximately located between the anode and cathode. The CEMs allow for the diffusion of the Na⁺ cations, and the BPMs allow the OH⁻ and H⁺ ions to migrate between the cathode and anode. Using the series of CEMs and BPMs proximately located between the anode and cathode, Na⁺ is allowed to diffuse into the cathode side of the cell, where it meets the OH⁻ anion to form NaOH in the base compartment; while SO²⁻₄ reacts with H⁺ generated from bipolar junction to form H₂SO₄ in the acid compartment. As illustrated in FIG. 11, the base compartment is the space between a CEM and an adjacent BPM proximately located on the cathode side of the CEM, and the acid compartment is the space between a CEM and an adjacent BPM proximately located on the anode side of the CEM. For purposes of clarification, it is noted that in contrast to employing EDBM, conventional electrolysis for water splitting reaction generates H₂ and O₂ gases, which consumes almost half the energy provided to the cell.

It is also noted that the function of an ion exchange membrane is to act like a thin selective barrier. Such membranes enable the electrically-driven selective transfer of ions between the two solutions, which they separate. Ion exchange membranes are composed of a polymer matrix on which are fixed ionized functional groups. These fixed charges are neutralized by mobile ions of opposite charge, called counter ions. Due to the Donnan effect, in an electrolyte solution, such membrane tends to reject ions with the same charge as the ionized groups, called co-ions. The cation exchange membranes (CEM) utilized in the EDBM exchange only cations between cathode and anode compartments, which is in contrast to anion exchange membranes (AEM) that exchange only anions between the electrode compartments in an electrolysis cell.

The bipolar membrane (BPM) utilized in the EDBM is composed of one cation-exchange layer and one anion-exchange layer joined together, which is used for water splitting. In contrast to cationic and anionic membranes, bipolar membranes have a required orientation between the electrodes: the anion-exchange layer should be oriented towards the cathode, and cation-exchange layer should be oriented towards the anode. If BPMs are placed with the wrong orientation, ions accumulate between the two layers resulting in blistering of the membranes. Unlike the conventional electrolysis, gas generation is minimized in the EDBM process due to membranes restricting the H⁺ and OH⁻ ions from reaching the electrode. Hence, theoretically the energy requirement is reduced to about 40% of what is required for water electrolysis. With an increase in number of unit cells or membrane stacks in the EDBM compartment, total energy consumption decreases, due to decrease in energy consumption in electrode compartment with minimized gas generation.

In some aspects, the scrubber solution provides a CO₂ capture efficiency of at least 90%, in some aspects at least 92.5%, in some aspects at least 95%, in some aspects at least 95.5%, in some aspects at least 96%, in some aspects at least 96.5% and in some aspects at least 97%.

In some preferred aspects, the scrubber solution comprises sodium hydroxide having a concentration between about 0.05 mol/L up to about 1 mol/L, preferably between about 0.075 mol/L up to about 0.75 mol/L, more preferably between about 0.1 mol/L up to about 0.5 mol/L, and in some preferred aspects preferably between about 0.25 mol/L up to about 0.4 mol/L.

The scrubber solution preferably captures CO₂ forming a captured CO₂ solution. In some aspects, the captured CO₂ solution comprises a sodium bicarbonate solution. In some preferred aspects, a scrubber solution comprising sodium hydroxide reacts with a flue gas to capture CO₂ forming the captured CO₂ solution comprising a sodium bicarbonate solution. Preferably the captured CO₂ solution is a sodium bicarbonate solution. In some preferred aspects, the captured CO₂ solution comprises at least 80%, in some aspects at least 85%, in some aspects at least 90%, in some aspects at least 95%, in some aspects at least 97.5%, in some aspects at least 98%, in some aspects at least 98.5%, in some aspects at least 99%, in some aspects at least 99.5%, in some aspects at least 99.9%, and in some aspects 100%, of a sodium bicarbonate solution.

In some aspects, the CO₂ is regenerated from the captured CO₂ solution such that a recovery rate of at least 90% is achieved, in some aspects at least 95%, in some aspects at least 98%, in some aspects at least 99%, in some aspects at least 99.5%, in some aspects at least 99.9%, and in some aspects essentially 100%.

In some aspects, the regenerated CO₂ produced from the captured CO₂ solution has a purity of at least 90%, in some aspects at least 95%, in some aspects at least 97.5%, in some aspects at least 98%, in some aspects at least 99%, in some aspects at least 99.5%, in some aspects at least 99.9%, and in some aspects essentially 100%.

The regenerated CO₂ is preferably produced by reacting the captured CO₂ solution with an acid reagent to provide the regenerated CO₂ and a resultant salt solution.

In some aspects, the scrubber solution is regenerated from the resultant salt solution by electrodialysis such that a regenerated scrubber solution yield of at least 90% is achieved, in some aspects at least 95%, in some aspects at least 98%, in some aspects at least 99%, in some aspects at least 99.5%, in some aspects at least 99.9%, and in some aspects essentially 100%.

In some aspects, the acid reagent is regenerated from the resultant salt solution by electrodialysis such that a regenerated acid reagent yield of at least 90% is achieved, in some aspects at least 95%, in some aspects at least 98%, in some aspects at least 99%, in some aspects at least 99.5%, in some aspects at least 99.9%, and in some aspects essentially 100%.

In some aspects, the scrubber solution and the acid reagent are both regenerated from the resultant salt solution by electrodialysis, such that a regenerated scrubber solution yield of at least 90% is achieved and a regenerated acid reagent yield of at least 90% is achieved, in some aspects at least 95%, in some aspects at least 98%, in some aspects at least 99%, in some aspects at least 99.5%, in some aspects at least 99.9%, and in some aspects essentially 100%, for both the regenerated scrubber solution yield and the regenerated acid reagent yield.

### EXPERIMENTAL

Continuous CO₂ capture and regeneration experiments were conducted on a mini pilot scale setup, as illustrated in the block diagram of FIG. 11, comprising a scrubber having scrubbing solution and a gaseous mixture inlet, an acid/base reaction tank in fluid communication with the scrubber, and an EDBM cell in fluid communication with the scrubber and also the acid/base reaction tank. As provided in FIG. 11, the NaHCO₃ solution formed by the capture of CO₂ by the scrubbing solution in the scrubber is fed to the acid/base reaction tank. Clean CO₂ is regenerated in the acid/base reaction tank by reacting the NaHCO₃ solution with an acid and also forming a resultant salt solution. The resultant salt solution is fed to the EDBM cell, wherein the acid and base are separated thereby regenerating a scrubbing solution for the scrubber and an acid for the acid/base reaction tank.

### Materials and Methods

CO₂ Absorption with NaOH

The scrubber column shown on the left side in FIG. 11 was used as a counter-current packed-bed absorption column. Column dimensions: Height: 275 cm; Diameter: 10.16 cm; Packing: Polypropylene pall rings 1.2 cm×1.2 cm; Packed bed height: 122 cm. To simulate flue gas, a gaseous mixture containing 16% volume CO₂ and the remaining 84% air was continuously fed into an air inlet proximately located the bottom of the scrubbing column with the help of a gas diffuser. Gas flow rate was maintained at 25 LPM. Separate flow meters were installed for CO₂ and air to measure the volumetric flow and to control the percentage of CO₂ in the gas stream. CO₂ and air flow rates were measured with gas flow meters (OMEGA) equipped with gas controllers (McMaster-Carr).

The percentage CO₂ of the simulated flue gas exiting out from the top of the column was measured with Quantek Model 906 infrared gas analyzer calibrated with a 20-vol % CO₂/N₂ reference gas. CO₂ capture efficiency of a NaOH solution as the scrubbing solution was measured by continuously recording percentage CO₂ absorption data by the data logger connected to the gas analyzer. After each experiment the data logger was connected to the computer and the graph generated from it was integrated to calculate the total moles of CO₂ absorbed per minute. The accuracy of the data was ensured by repeating these experiments in triplicates. For a 16% CO₂ gas stream (simulating a power plant flue gas), the optimum parameters were found to be: 0.3 mol/L NaOH solution at 6.4 Liters per minute flow rate.

Scrubber Solution Regeneration with EDBM

The scrubber solution, particularly a NaOH solution, was regenerated through an EDBM unit as shown in FIG. 11. The electrodialysis setup shown in FIGS. 12A-12B consisted of a DC power supply (XHR40-25, AMETEK; 0-40 V, 0-25A) to maintain constant current field. The electrodialysis cell components and membrane stack were obtained from Ameridia—The Eurodia Group (properties given in Table 5). Membranes were separated by 0.8 mm thick spacers. To maintain the same pressure between acid, salt, and base compartments, pressure gauges (15 psi max) were installed. Volumetric flow was measured with flow meters (OMEGA). The EDBM unit was equipped with instruments to measure conductivity, voltage, current and temperature.

TABLE 5
Properties of membranes used in EDBM stack.

| | | Area | Burst | Selec- | Effi- |
|---|---|---|---|---|---|
| | Thickness | resistance | strength | tivity | ciency |
| Membrane | (mm) | (Ω · cm²) | (kPa) | (%) | (%) |
| CBM cation- | 0.21 | 4.5 | ≥400 | >98 | — |
| exchange membrane | | | | | |
| Neosepta ® | 0.22 | — | ≥400 | — | >98 |
| bipolar membrane | | | | | |

In the setup illustrated in FIG. 11, after CO₂ is absorbed in the scrubber column, the absorbent solution (NaHCO₃) is reacted with H₂SO₄ solution in the acid/base reaction tank, and the resultant Na₂SO₄ solution is fed into the electrodialysis cell. The salt solution (0.2 M Na₂SO₄) was prepared by mixing Na₂SO₄ in water. Na₂SO₄>99% reagent grade was obtained from Sigma-Aldrich. To ensure an initial conductivity greater than 20 mS/cm, acid and base tanks were mixed with H₂SO₄ and NaOH, respectively. NaOH>98% reagent grade was obtained from Sigma-Aldrich. 98% w/w H₂SO₄ was obtained from Fisher Scientific. NaOH concentration from the base compartment was measured by titration with 0.01 mol/L HCl standard solution. Acid concentration was estimated by measuring the pH constantly with Oakton 150 hand held pH meter. The solution in the acid/base reaction tank was continuously stirred with an immersion drum mixer. The experimental conditions used for the setup shown in FIG. 11 are provided in Table 6. For the idling procedure, each compartment was filled with deionized water. If the idle time lasted more than a day, each compartment was filled with salt solution at 30 g/L (50 mS/cm conductivity).

TABLE 6
Operating parameters for the experimental setup shown in FIG. 11.

| Experimental Conditions | |
|---|---|
| Scrubbing liquid to gas ratio (L/G:Kg/Kg) | 4.3 |
| Gas composition | 16% vol. CO₂, |
| | remaining air |
| Gas inlet temperature (° C.) | 31 |
| EDBM cell volume (m³) | 0.012 |
| EDBM cell voltage (V) | 10-20 |
| Current (A) | 1-16 |
| Temperature (° C.) | 30 |
| Pressure (kPa) | 101.32 |
| Initial conductivity of acid/salt and | 20 |
| base compartment (mS/cm) | |
| Maximum conductivity (mS/cm( | 220 |
| Single membrane area (m²) | 0.04 |

### Experimental Procedure

CO₂ capture and regeneration experiments were conducted with the setup shown in FIG. 11. Before running this setup in continuous mode, CO₂ capture experiments were conducted in the scrubber with different concentrations of NaOH. Concentrations of 0.1-0.4 mol/L were tested and a maximum CO₂ capture efficiency of 97% was observed at concentrations of 0.3 mol/L and higher. After finding that 0.3 mol/L was the optimum concentration for achieving maximum absorption efficiency, the EDBM setup was run for 30 min to achieve desired NaOH concentration.

Before running and regeneration setup in continuous mode with the capture column, EDBM cell was run for 30 minutes until the desired acid and base concentrations were reached, starting with 0.2 mol/L Na₂SO₄, 0.1 mol/L NaOH and 0.02 mol/L H₂SO₄ concentrations. Acid and base concentrations were started at 0.024 mol/L and 0.1 mol/L, respectively, to ensure the initial conductivity of the cell was greater than 20 mS/cm, for proper functioning of EDBM. Several voltage ranges were tested for the EDBM cell, and for each constant voltage, current density was recorded every minute until it reached a maximum value. Then the setup shown in FIG. 11 was run in continuous mode for 3 hours to ensure no discrepancy in CO₂ capture and regeneration. CO₂ absorption data was continuously recorded by the gas analyzer for the entire duration of the experiment. CO₂ absorption was continuous at 97% absorption efficiency throughout the duration of 3 hours. Each experiment was repeated three times to ensure reproducibility.

### Results and Discussion

CO₂ Absorption with NaOH

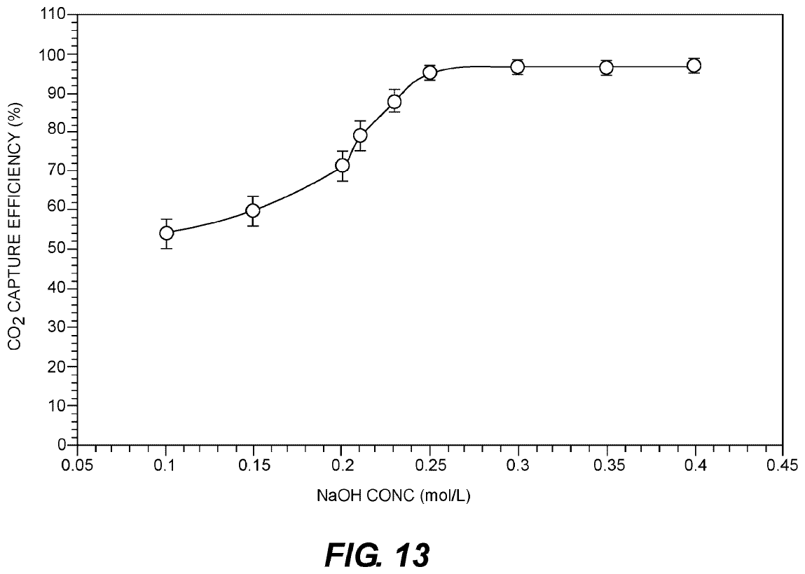

The CO₂ absorption efficiency of NaOH solution at various concentrations is shown in FIG. 5. Initial absorption experiments were conducted with NaOH concentration ranging from 0.1 mol/L to 0.4 mol/L. As shown in FIG. 13, the absorption efficiency of the solution slowly increased with increasing NaOH concentration, finally reaching an asymptote after 0.3 mol/L at 97% capture efficiency.

NaOH Regeneration with EDBM

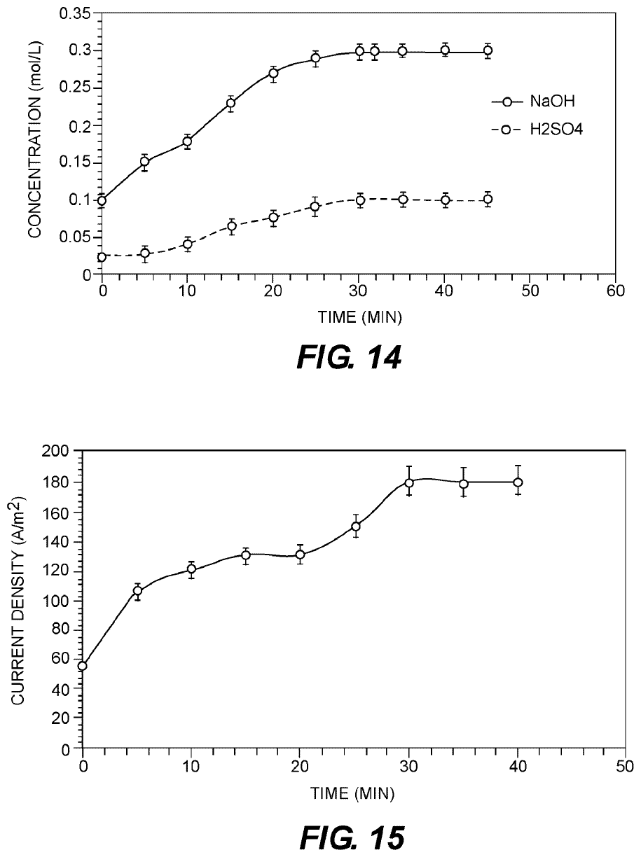

Initial batch tests were conducted on EDBM cell for 30 minutes until the desired acid and base concentrations were reached. The maximum acid and base concentrations were reached in about 30 minutes as shown in FIG. 14. FIG. 14 shows an increase in acid and base concentration with time, until both the acid and base reach asymptote after about 30 minutes, then the continuous CO₂ capture and regeneration experiments were run for 3 hours with constant current intensity. As shown in FIG. 15, at constant voltage the current density increased with an increase in time, acid and base concentration due to an increase in conductivity. Once maximum conductivity is reached, the cell operates at a constant current intensity for a given voltage. All the experiments were repeated through three independent measurements. The experimental uncertainty was calculated, and the results were plotted within 95% confidence interval.

Comparing Two Compartment Configuration with Three Compartment Configuration

FIGS. 12A-12B show additional detail of the two compartment EDBM configuration used in the systems of FIGS. 10 and 11 of the present disclosure. The two-compartment configuration has BPM and CEM as the repeating unit cell. The number of repeating unit cells can be greater than 1 and up to 100 or more, and contemplated to be any subset within the foregoing range. In this system, the number of repeating unit cells was 7. In three compartment configurations, the repeating unit cell has AEM, CEM and BPM in respective order. Three compartment configurations are generally used for creating higher concentration of both acid and base. Three compartment configurations tend to create concentrations of more than twice that of two compartment cells. In the present experiment, dilute concentrations of acid and base are required, such that a two-compartment configuration is advantageous in achieving high current efficiency at low voltage ranges. It has also been previously observed that a two compartment EDBM configuration reaches desired maximum acid and base concentrations in less time (within first 60 min) compared to three compartment configurations (few hours). In the present experiment, the maximum acid and base concentrations were reached in about 30 minutes as shown in FIG. 14. Although, this time is only significant during the batch testing, in continuous mode two compartment configuration consumes less energy due to lower acid and base concentrations required for the present system.

### Effect of Current Density on Energy Consumption, Current Efficiency and NaOH Concentration

Since the energy consumption was the primary focus, the effect of current density on energy consumption was studied. Current density and energy consumption also have a direct influence on NaOH concentration, which in turn has an effect on CO₂ capture efficiency. With increase in the concentration of electrolyte solution (0.2 M Na₂SO₄), the current efficiency decreases. Also, higher concentration leads to high osmotic pressures and reduction in water dissociation at the bipolar membrane.

### Current Efficiency

Current efficiency defines how effectively the ions are transported across the membranes. Current efficiency decreases as the electrolyte and base concentrations increase. A low current efficiency may also result from the imperfect orientation of the membranes that allow the transfer of some co-ions, particularly when the concentrations are higher. Current efficiency is calculated from Equation (35).

$$η = \frac{z \cdot F \cdot V ( {C}_{t} - {C}_{0} )}{n \cdot I \cdot t} \qquad ( 35 )$$

where n is the number of cells (repeating membrane units; n=7 in the present experiment), V (L) is the circulated volume of the solution, F is Faraday's constant (96,500 coulombs/mol) C₀ and Cₜ are the concentrations (mol/L) of NaOH at time 0 and time t, z=1 in the present situation with OH⁻ carrying unit negative charge, and I (A) is the current across the cell.

### Energy Consumption

Total energy consumption in kWh Kg⁻¹ of CO₂ captured is calculated from Equation (36). This energy is converted to MJ Kg⁻¹ by multiplying with a conversion factor of 3.6.

$$Energy consumption = \int \frac{U \cdot I \cdot dt}{{V}_{t} \cdot {C}_{t} \cdot M} \qquad ( 36 )$$

wherein U (V) is voltage across EDBM cell, I (A) is the current across the cell, Cₜ is the concentration of CO₂ at time t, Vₜ (L) is the volume of the solution circulating through the setup, and M is the molecular weight of CO₂ (44.01 g/mol).

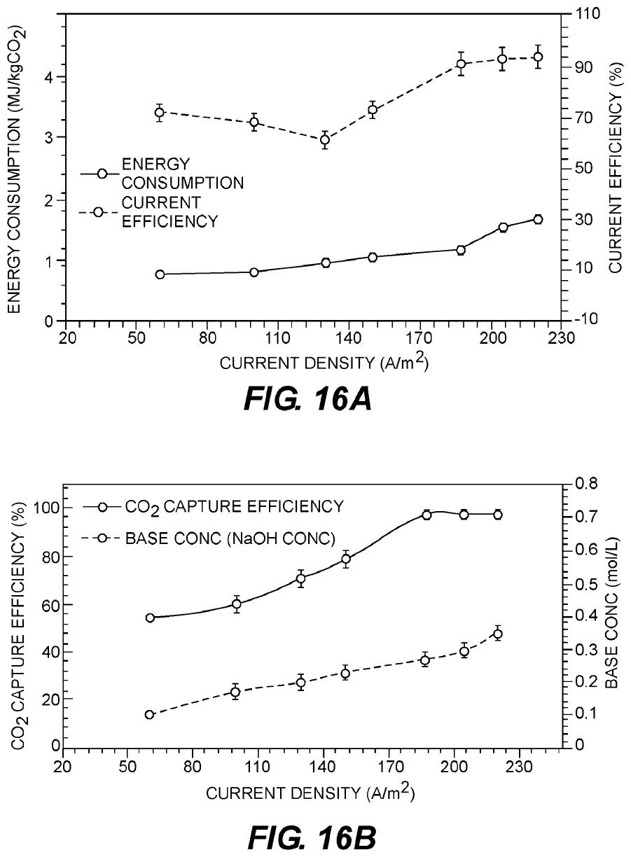

FIG. 16A shows the effect of current density on energy consumption and current efficiency. Current efficiency initially decreases with increase in current density because of low ion selectivity of membranes at lower ranges of current density, but current efficiency starts to increase once the current density is over 140 A/m² due to higher ion transport in the base compartment because of higher conductivity. Increase in current density from 150 A/m² to 180 A/m² only increases the energy slightly from 1.03 to 1.18 MJ, but this increase is more pronounced from 190 A/m² to 200 A/m² due to increase in base concentration at peak current (I) from Equation (36). Increasing the base concentration by more than 0.3 mol/L increased the current efficiency, but it also increased the overall energy consumption. Considering the total energy consumption as the criteria for the overall process, it would be desirable to stay below the current density of 180 A/m².

Decrease in current efficiency was observed at lower current density and base concentrations. In the two-compartment configuration the hydron (H⁺), which migrates through the cation exchange membrane and recombines with hydroxide ion (OH⁻), slightly reducing the current utilization. The energy consumption is less in case of two compartment configurations because of dilute base concentration, while that of acid concentration effect on energy consumption follows the opposite trend, as previously observed by others when conducting studies on recovery of H₂SO₄ from Na₂SO₄ salt solution.

FIG. 16B indicates that as the current density increases, the base concentration keeps increasing, but the CO₂ capture efficiency reaches a plateau at 97% capture efficiency. Increasing the base concentration further will leave unreacted NaOH in the captured solution. Further increasing the base concentration will increase the energy consumption of EDBM cell at higher current densities as shown in FIG. 16A. So, the optimum values for the current density and base concentration are: 181.7 A/m² and 0.3 mol/L respectively, keeping the energy consumption minimum and achieving 97% CO₂ capture efficiency. Therefore, the optimum operating conditions of the cell are: 18 V, 7.5 A.

### Performance Evaluation

The regeneration energy of 1.18 MJ/KgCO₂ when compared to 3-4 MJ/KgCO₂ in case of thermal regeneration with amines and other absorbent solutions is a huge breakthrough in terms of energy savings. In the case of thermal regeneration, evidence suggests that an increase in stripper energy from 3 MJ/KgCO₂ to 4 MJ/KgCO₂ will reduce the power plant output by at least 20%. The important advantage of using EDBM process is that renewable sources like photovoltaics can be used to energize the EDBM cell. Some researchers who worked on direct electrodialysis of NaHCO₃ solution were able to achieve low energy values (2-3 MJ/KgCO₂) compared to thermal regeneration, but the direct electrodialysis process has its own fair share of process complications as mentioned above, and more importantly very low CO₂ recoveries (40-60%).

As opposed to direct electrodialysis, an important trait in the presently disclosed process is the greater than 60%, in some aspects greater than 70%, in some aspects greater than 80%, in some aspects greater than 90%, in some preferable aspects greater than 95%, in some other preferable aspects greater than 98%, in some even more preferable aspects greater than 99%, in some aspects up to 99.5%, in some aspects up to 99.6%, in some aspects up to 99.7%, in some aspects up to 99.8%, in some aspects up to 99.9%, in some aspects up to 99.99%, and in the most preferable aspect up to 100%, recovery rate of CO₂.

Since all the CO₂ is essentially capable of being recovered prior to the EDBM step, the system and method of the present disclosure eliminates the presence of gas bubbles in the cell, avoiding unnecessary resistance across the cell. Hence, a very high current efficiency of 91% was observed, due to relatively lower concentration of acid and base generated. It is hypothesized that the limitation in current efficiency could be due to the leakage of protons through the cation exchange membrane. In a commercial scale EDBM unit, the number of unit cells could be much larger as opposed to lab scale unit of the present experiment, in which case voltage drop across the EDBM stack would be much less at lower current densities. Thus, the energy requirement is anticipated to be further lowered in a commercial scale unit.

An advantage of the system and regeneration method of the present disclosure is that the regeneration can be performed at room temperature and atmospheric pressure conditions, as opposed to high pressures required for direct electrodialysis of NaHCO₃ as mentioned earlier. This ensures high process safety and also easier start-up and shutdown.

In some aspects, a particulate filtration step is recommended before the scrubber to ensure no particulates enter the EDBM cell. The usual norm in industrial flue gas capture is to remove/filter suspended particulates before sending the gas for flue gas desulfurization (FGD) and subsequently CO₂ capture. Accordingly, the system and method of the present disclosure may in some aspects having a particulate filter and particulate filtration step prior to the scrubber in order to avoid suspended solids going into the EDBM cell and fouling membranes.

### Cost Estimation

Economic analysis was also carried out by considering a hypothetical case of 400 MW coal-fired power plant, which corresponds to 300° tons/h of CO₂ emissions and a continuous operation of CO₂ capture and regeneration for 350 days a year at 24 h per day. With 97% CO₂ capture efficiency of NaOH from the foregoing experimental results, this accounts to 2.4 Mton/year of CO₂ captured. All assumptions for cost estimation are shown in Table 7.

TABLE 7
Operating parameters and assumptions made for cost
estimation of CO₂ capture with EDBM regeneration.

| Parameter | | Life Span | |
|---|---|---|---|
| Number of EDBM cells | 2867 | 15 | years |
| Bi-polar membrane price ($/cm²) | 0.43 | 5 | years |
| Cation exchange membrane price ($/cm²) | 0.24 | 5 | years |

| | | |
|---|---|---|
| Electricity cost ($/kWh) | 0.06 | — |
| Cost of each EDBM stack ($/cm²) | 1.005 | — |

Considering the foregoing base case scenario for capital cost, the operating cost is estimated by calculating the direct energy cost.

### Cost of CO₂ Absorption

Cost of CO₂ capture with thermal regeneration from previous literature was estimated to be around 45-60$/ton of CO₂ captured, and others estimated that 30% of this cost corresponds to CO₂ absorption equipment, which includes absorption column and pumping system. Considering the same base case scenario, the CO₂ absorption cost is estimated to be 13.5$/ton of CO₂ captured.

### Cost of CO₂ Regeneration

Cost of CO₂ regeneration or reagent regeneration was estimated based on laboratory results of EDBM experiments. The cell in the lab has a cell volume of 0.012 m³ and handles 7.5 L/min of solution. At the L/G ratio of 4.3 and gas flow of 5 tons/min the total liquid to be handled by EDBM cells is 21500 LPM. Therefore, the number of cells required are 2867. Cost of each EDBM stack was estimated as 1.5 times the cost of membranes, based on previous work in the industry. Total equipment cost and operating costs are provided in Table 8.

TABLE 8
Total equipment costs (TEC) and operating costs.

| Capital costs | (Million $) |
|---|---|
| Cost of EDBM cells (including membrane cost) | 26.62 |
| Other equipment* | 28.5 |
| TEC | 55.12 |

| Operating costs | $/ton of CO₂ captured) |
|---|---|
| Energy cost | 19.62 |
| Labor and maintenance (13% of Investment Cost) | 0.52 |
| Other variable costs** | 0.39 |

*Other equipment includes spacers, pipelines, pumps, CO₂ compression etc., as estimated by the work of Sabatino et al. (2020).
**Other variable costs include pumping costs and other miscellaneous expenses.

Total capital cost including equipment cost, construction, valves, piping, etc. is calculated based on NETL guidelines as provide in Table 9.

TABLE 9
Total capital cost considerations.

| | |
|---|---|
| Total installation cost (TIC) | 80% TEC |
| Total direct plant cost (TDPC) | TEC + TIC |
| Indirect costs (IC) | 13% TDPC |
| Engineering, procurement and construction (EPC) | TDPC + IC |
| Total contingencies and owner's cost (C&OC) | 30% EPC |
| Total capital (TC) | EPC + C&OC |

The total capital investment is about 145.73 M$ for 15 years of operation and 36 Mtons of total CO₂ processed. As such, for 1 ton of CO₂, captured the total capital investment turns out to be about 4.04$/ton of CO₂ captured. If both capital expenditure and variable operating costs are combined, the total cost of CO₂ capture and regeneration would be 38.07$/ton of CO₂ captured. Although the operating costs are very low, the capital cost increases the total cost due to high EDBM unit prices and membrane prices. Membrane prices are expected to go down further in the future, in such a case the total cost can be less than 38.07$/ton of CO₂.

Depending on the project timeline, EDBM will be advantageous if the project period is extended over 15 years. It can also be made profitable over a shorter period of time if the membrane prices are lowered. Further decreases in electricity costs may also be anticipated by 2050, with developments in renewable energy technologies.

An additional benefit of the EDBM method is that you can regulate the base concentration as required by adjusting the voltage and current across the cell. If the CO₂ concentration from the flue gas is fluctuating due to load variation from the power plant, this turning might help reduce the cost on daily basis. Considering 15 years of project timeline, the average cost per ton of CO₂ captured is roughly 38$. It is contemplated that the reagent regeneration energy of 1.18 MJ/kg could be further reduced with numerous performance improvements and careful design choices, further making CO₂ capture economically feasible and environmentally benign.

As provided by the foregoing, the present inventors have developed a new regeneration method for CO₂ capture with an alkali absorbent solution, whereby sodium bicarbonate is reacted with an acid, preferably sulfuric acid, and the resultant salt solution, preferably a sodium sulphate solution, is subjected to an EDBM process for regenerating the alkali absorbent solution, preferably NaOH, and the acid. The present inventor were able to achieve reagent regeneration energy as low as 1.18 MJ/kg of CO₂ captured at a current efficiency of 91.2% for the EDBM cell. The cost of processing flue gas is around 38.07$/ton of CO₂ captured based on 2020 prices. This cost could be even lower if membrane costs were competitive. As such, the system and process of the present invention provides a very promising choice for post-combustion CO₂ capture.

### IV. Electrochemical Approach for Converting Carbon Dioxide to Oxalate

With increased CO₂ emissions into the atmosphere, there is great opportunity to capture CO₂ and utilize the captured CO₂ for economic advantage. Developing energy-efficient processes that reductively couple CO₂, an abundant and renewable carbon source, for the production of value-added chemicals (methanol, ethanol, and oxalic acid) using electrochemical processes is a goal of great importance. In many cases, these chemicals can be reused elsewhere in the refining process or sold as valuable byproducts.

Electrochemical reduction of CO₂ to oxalic acid and other chemicals is a complex multistep reaction with adsorbed intermediates. The present inventors are not currently aware of the exact reaction mechanism for the electrochemical reduction of CO₂ to oxalic acid, which is dependent upon a range of conditions like electrode type, electrode potential, Current density, catalyst, etc. The present inventors have successfully produced oxalic acid from CO₂ with the help of electro-catalytic reduction, and the results are discussed in this section.

The large contribution in total CO₂ emission originates from coal or natural gas power plants, and a considerable amount from steel plants. Capturing the available CO₂ from the steel and coal industries for economic advantage is a win-win situation for the industry. This technology is not only important scientifically but is also vital for a sustainable future. The various ongoing investigations can be categorized as biochemical, thermochemical, photochemical, and electrochemical approaches. Among these, the electrochemical method shows the most promise as an efficient form of CO₂ conversion technology, because of its many advantages like high reactivity under ambient conditions and good extensibility from small- to large-scale processes.

CO₂ is thermodynamically quite stable, as shown by its highly negative heat of formation. Thus, it is expected that the formation of any useful chemical from CO₂ will require the input of at least as much energy as geological sequestration. This lends itself to two extremes: one where the quantity of energy required is low and one where the economic value of the additional energy is low.

It is expected that CO₂ can be reduced via electrolysis to several compounds. In certain aspects, the captured CO₂ is converted to one or more desired chemicals. In certain aspects, the one or more desired chemicals derived from captured CO₂ is formic acid, oxalic acid, methanol, ethanol, formaldehyde, and carbon monoxide (as a component to syn-gas).

The present inventors have discovered an electrochemical reduction of captured CO₂ to an oxalate salt. The present inventors have also prepared oxalic acid from an oxalate salt formed by the electrochemical reduction of captured CO₂.

In some preferred aspects, the captured CO₂ utilized in the electrochemical reduction process to produce an oxalate salt and/or oxalic acid is purified CO₂. In some preferred aspects, the captured CO₂ is industrial grade having a purity of at least 99.5%. In some preferred aspects, the captured CO₂ is medical grade having a purity of at least 99.5%. In some preferred aspects, the captured CO₂ is bone dry grade having a purity of at least 99.8%. In some preferred aspects, the captured CO₂ is food grade having a purity of at least 99.9%. In some preferred aspects, the captured CO₂ is beverage grade having a purity of at least 99.9%. In some preferred aspects, the captured CO₂ is anaerobic grade having a purity of at least 99.95%. In some preferred aspects, the captured CO₂ is research grade having a purity of at least 99.999%.

In some preferred aspects, the captured CO₂ is captured from flue gas and has undergone processing to a purity of at least 99.5%, is some aspects at least 99.8%, in some aspects at least 99.9%, in some aspects at least 99.95%, and in some other aspects at least 99.999%.

In some preferred aspects, the captured CO₂ is from flue gas that has been captured using an absorption column, in some preferred aspects the chemical absorption capture of CO₂ using a scrubbing absorption column containing a slurry solution having a frothing agent as previously disclosed in Section I. In some preferred aspects, the captured CO₂ has a purity of at least 99.5%, is some aspects at least 99.8%, in some aspects at least 99.9%, in some aspects at least 99.95%, and in some other aspects at least 99.999%.

In some preferred aspects, the captured CO₂ is from flue gas that has been captured using a single stage absorption column, in some preferred aspects the chemical absorption capture of CO₂ using a scrubbing absorption column containing a single wet scrubbing absorption column at alkaline pH conditions for the simultaneous capture of CO₂, NOₓ and SOₓ from flue gas as previously disclosed in Section II. In some preferred aspects, the captured CO₂ has a purity of at least 99.5%, is some aspects at least 99.8%, in some aspects at least 99.9%, in some aspects at least 99.95%, and in some other aspects at least 99.999%.

In some preferred aspects, the captured CO₂ is from flue gas that has been captured and purified by thermal regeneration as previously disclosed in Section III. In some preferred aspects, the captured CO₂ has a purity of at least 99.5%, is some aspects at least 99.8%, in some aspects at least 99.9%, in some aspects at least 99.95%, and in some other aspects at least 99.999%.

In some preferred aspects, the captured CO₂ is from flue gas that has been captured and purified by the EDBM system and process as previously disclosed in Section III. In some preferred aspects, the captured CO₂ has a purity of at least 99.5%, is some aspects at least 99.8%, in some aspects at least 99.9%, in some aspects at least 99.95%, and in some other aspects at least 99.999%.

In some aspects, a cathode surface is modified for the absorbing and conversion of captured CO₂ into an oxalate salt via an electrochemical reduction. In some preferred aspects, the cathode is wrapped at least partially around the anode in a cylindrical configuration. In some aspects, the cathode is wrapped around the anode in a cylindrical configuration allowing for the elimination of the membrane typically used to separate the catholyte and anolyte region.

In some aspects, a cathode surface is modified with a coating for the absorbing and conversion of CO₂ into an oxalate salt via an electrochemical reduction. In some preferred aspects, the cathode surface is modified with a metal coating that provides a rough surface area compared to the cathode without the metal coating, such that the metal coating increases the surface area of the cathode surface. In some preferred aspects, the metal coating is a lead coating, a zine coating or a steel coating.

In some preferred aspects, the cathode comprises zinc and the metal coating comprises a lead coating, a zine coating or a steel coating. In some preferred aspects, the metal coating increases the surface area of the cathode by providing a rough surface area compared to the cathode surface without the metal coating.

In some preferred aspects, the electrochemical reduction of captured CO₂ to the oxalate salt occurs in the presence of an aprotic solvent. In some preferred aspects, the electrochemical reduction of captured CO₂ to the oxalate salt occurs in the presence of an aprotic solvent with at least one catalyst, in some preferred aspects an electrocatalyst. In some preferred aspects, the catalyst comprises an aromatic nitrile catalyst. In some preferred aspects, the aromatic nitrile catalyst comprises O-tolunitrile (2-methyl benzonitrile). In some other preferred aspects, aromatic esters, aromatic nitriles and transition metal complexes are anticipated to be efficient electrocatalysts in the electrochemical reduction transformation of captured CO₂ to one or more oxalate salts. Without wishing to be bound be theory, it is believed that electrochemically generated anion radicals of aromatic nitriles and/or aromatic esters are capable of reducing captured CO₂ to oxalate with negligible formation of carboxylated products in an apoptic solvent.

In some aspects, the catalyst is chose from the group consisting of dimethyl phthalate, diisobutyl phthalate, dibutyl phthalate, methyl 4-phenylbenzoate, phenyl benzoate, phenyl 3-methylbenzoate, ethyl 3-fluorobenzoate, methyl 3-phenoxybenzoate, phenyl 4-methylbenzoate, methyl benzoate, ethyl benzoate, methyl 3-methylbenzoate, methyl 2-methylbenzoate, methyl 4-methylbenzoate, 4-cyanobiphenyl, benzonitrile and O-tolunitrile.

The conversion of captured CO₂ to the oxalate salt has at least a 50% coulombic efficiency, in some aspects at least a 50% coulombic efficiency, in some aspects at least a 55% coulombic efficiency, in some aspects at least a 60% coulombic efficiency, in some aspects at least a 65% coulombic efficiency, in some aspects at least a 70% coulombic efficiency, in some aspects at least a 75% coulombic efficiency, and in some aspects at least a 80% coulombic efficiency.

The conversion of captured CO₂ to the oxalate salt has up to 80% coulombic efficiency, in some aspects up to about 85% coulombic efficiency, in some aspects up to about 87.5% coulombic efficiency, in some aspects up to about 90% coulombic efficiency, in some aspects up to about 92.5% coulombic efficiency, in some aspects up to about 95% coulombic efficiency, in some aspects up to about 97.5% coulombic efficiency, and in some aspects up to about 99% coulombic efficiency.

In some preferred aspects, the voltage during the electrochemical reduction is between about 6 and about 11 volts with a current density of more than 25 mA/cm².

### EXPERIMENTAL

Electrocatalytic Production of Oxalate from CO₂

### Initial Oxalate Production Using Membrane Electrolysis Cell

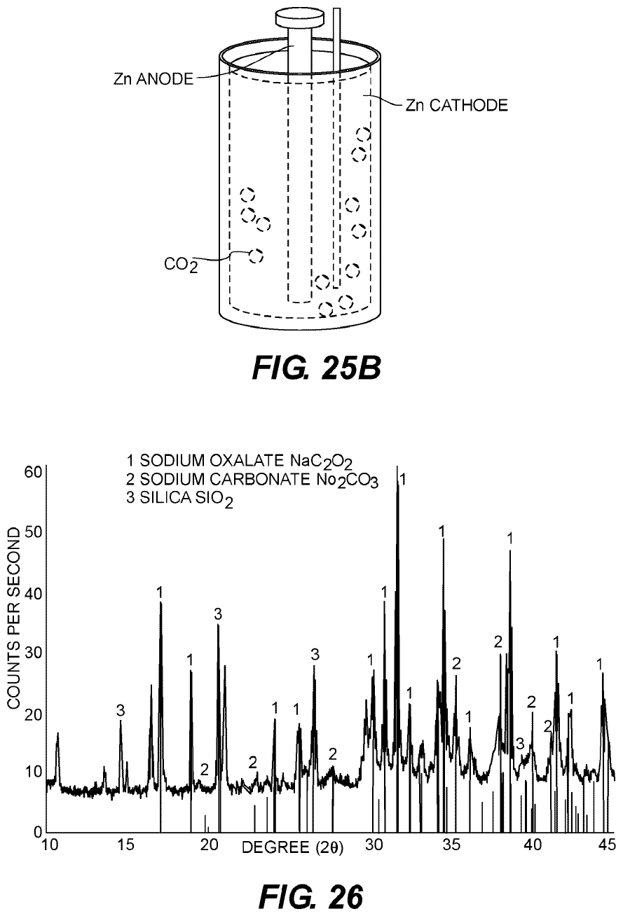

A membrane electrolysis cell was initially used to produce oxalate from CO₂. A membrane electrolysis cell is a 2-chamber electrolysis cell whereby the chambers are separated by a selectively permeable membrane. To produce oxalate, a cation exchange membrane (which selectively exchanges cations) was used. Literature suggests that in the cathode chamber an organic electrolyte, such as tetraethylammonium perchlorate or tetraethylammonium bromide (TEA-Br) in dimethylformamide (DMF), are preferred. In the anode chamber, a sodium hydroxide solution water was used. Carbon dioxide was bubbled into the cathode chamber as a current was applied. This described process is illustrated in FIG. 25A.

### Catholyte and Anolyte Concentrations:

Catholyte: DMF, 0.1 M TEA-Br, 0.01 M o-tolunitrile. Anolyte: Water, NaOH buffered with sodium bicarbonate to a pH of 9.8. This catholyte and anolyte composition was utilized in the process shown in each of FIGS. 25A and 25B.

### Oxalate Production Using Modified Electrode Surface

The electrolysis cell was modified for improved cathode surface area to adsorb more CO₂ for more conversion. In particular, the cathode was wrapped in a cylindrical configuration around the anode, keeping the total cell volume constant, as shown in FIG. 25B. This way we can eliminate the membrane separated catholyte and anolyte region. The cathode surface as also modified with a lead coating. Lead has proven more selective for producing oxalic acid in an aprotic solvent. The lead coating on the cathode surface provides a rough surface, which provides more active sites for CO₂ to adsorb and undergo further steps of reduction. In this particular experiment, both the anode and the cathode comprised zinc with the cathode surface area being 100 cm³ and the cell volume being 150 mL. Carbon dioxide was bubbled into the cathode chamber as a current was applied. A solid precipitate formed within the cell.

### XRD Studies

The solid precipitate sample from the modified electrolysis cell was dried in a vacuum drying oven and hand ground for XRD analysis. X-ray Powder Diffraction was used to identify different phases in the solid precipitate sample collected from experiments. The XRD pattern of the solid sample was determined by using Scintag XDS2000 Powder Diffractometer in a 20 range of 10-45° at a scanning rate of 2.4° min⁻¹.

### Results and Discussions

Generation of oxalate from CO₂ without the addition of a catalyst is thermodynamically unfavorable at 298K and atmospheric pressure, due to the high negative redox potential (E=−2.2V vs SCE). The addition of an aromatic nitrile, such as O-tolunitrile, as a catalyst results in a highly selective reaction system. This should create sodium oxalate with 80% or higher coulombic efficiency. Without the catalyst, the reaction is more favored towards carbon monoxide product.

The primary reaction is the electron addition to an aromatic nitrile catalyst A+e⁻→A^{·-}, which is accompanied by electron transfer to CO₂ from anion radical A^{·-}+CO₂→A+CO₂^{·-}, which then dimerizes to oxalate 2CO₂^{·-}→C₂CO₄²⁻. The oxalate was collected at the bottom of the cell as zinc oxalate solid precipitate. It was also found that the cation exchange membrane is not intended for use in strongly basic solutions. The buffering with a sodium hydroxide solution in the anolyte region to a lower pH with a weak acid eliminated the negative effects on the exchange membrane.

### Electrode Selectivity

Type of electrode and cell potential can play an important role in electrochemical reduction of CO₂. Lead and steel can be mentioned as good examples of inert, “outersphere” electrode materials for CO₂ reduction. Lead, zinc and steel electrodes with rough surface have shown promising results. The present inventors tested a range of voltages and current densities. In some preferred aspects, the voltage of 6 to 11 volts and a current density of more than 25 mA/cm² provided promising results, as opposed to less than 3 volts and 25 mA/cm². FIG. 26 shows the XRD of an oxalate sample produced at 11 volts. As shown in FIG. 26, high intensity oxalate peaks were observed when the voltage and current density were 11 volts and 25 mA/cm². The present inventors investigated lead, copper, zinc, silver and steel as cathode materials. Zinc and steel have proven to be most efficient among others, which without wishing to be bound by theory is believed to be due to an alloying effect. Table 10 shows the weight percent of oxalate produced at different cathode materials and various voltages. When gases accumulate on the cathode surface over time, the reduction potential decreases. In this scenario, most of the energy provided is lost as heat. When this happens the reaction-rate slows down and eventually stops. To avoid the reaction rate from slowing down and stopping, the electrolyte was continuously stirred.

TABLE 10
Oxalate product obtained in weight percent
at different cathode materials.

| Cathode | Cell Potential | | Final product (Individual wt %) |
|---|---|---|---|
| Zinc | 3.5 | V | Negligible solid precipitate |
| Steel | 6 | V | Oxalate (1.2%), rest zincite |
| Silver | 11 | V | Oxalate (5%), Zincite (ZnO) |
| | | | powder (50%)*, TEABR (25%) |
| Steel | 11 | V | Oxalate (91%), zincite (6), |
| | | | TEABR (3%) |
| Zinc | 11 | V | Oxalate (95%), formate (3%), |
| | | | TEABR (2%) |
| Lead | 11 | V | Oxalate (75%), formate (10%), |
| | | | zincite (10%) |
| Iron | 11 | V | Oxalate (45%), zincite (25%), |
| (Mild steel) | | | TEABR (15%) |
| Copper | 11 | V | Oxalate (86%), formate (2%), |
| | | | zincite (10%) |

Table 11 shows coulombic yield vs. current density observations for different cathode materials tested. As one can observe, lead, Zinc and steel have more catalytic activity for generating oxalate from CO₂. In addition, steel electrode coated with lead surface irregularities has shown very high (85.23%) coulombic yield. Surface irregularities result in more active surface sites available for effective charge transfer.

Coulombic Yields were Determined as Follows:

$$Theoretical yield of Oxalate ( grams ) = \frac{Numbers of coulombs \times mol . wt . of oxalate}{2 \times 96 , 500 coulombs}$$

$$\begin{matrix}Percent coulombic yield = \frac{actual yield}{theoretical yield} \times 100 \\ = \frac{gm of oxalate salt}{theoretical yield of Oxalate \{ gm \}} \times 100\end{matrix}$$

TABLE 11
Current density vs coulombic yield observations.

| Cathode | Current density (mA/cm²) | % Coulombic yield* |
|---|---|---|
| Zinc | 10 | — |
| Steel | 15 | 5.23 |
| Silver | 25 | — |
| Steel | 25 | 87.23 |
| Zinc | 25 | 95.01 |
| Lead | 27 | 60.10 |
| Iron (Mild steel) | 25 | 59.32 |
| Copper | 25 | 85.23 |

### Redox Catalysis

In case of aromatic esters and nitrile catalysts, the reduction product is exclusively oxalate. When the standard potential of the catalytic pair is more positive, the catalytic efficiency decreases rapidly. These findings relate to a system of redox catalysis through which two CO₂ anion radicals would combine to produce oxalate after being generated by transferring an outersphere electron between CO₂ and the anion radical of nitrile or ester.

The reaction scheme involving aromatic nitrile catalyst ‘A’ is shown below. In step (39) the CO₂ anion radicals undergo dimerization to form oxalate anion. Step (3) is a fast reaction. In step (40) the addition product of carbon-oxygen formed from CO₂^{·-} and CO₂ is due to the base characteristic of CO₂^{·-} and lewis acid properties of CO₂. This intermediate step has been previously investigated by Seveant, et. al. (1983), to explain the formation of CO in competition with oxalate at electrodes with low hydrogen overpotential. The present inventors observed formation of sodium carbonate (shown in XRD image in FIG. 26) along with oxalate, which confirms the mechanism observed in step (41).

| | |
|---|---|
| A + e^{•} [symbol] A^{•} ⁻ | (37) |
| A^{•} ⁻ + CO₂ [symbol] A + CO₂^{•} ⁻ | (38) |
| 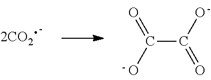 | (39) |
| 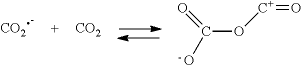 | (40) |
| 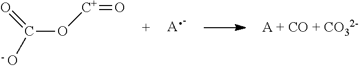 | (41) |

Table 12 shows the catalysts considered in the electrochemical reduction of captured CO2 in the presence of an aprotic solvent, which includes the standard potentials and rates constants of the reaction with CO2. The catalyst O-tolunitrile was selected in the present experiments.

TABLE 12
Homogeneous Catalysts.

| | E°cat | log k |
|---|---|---|
| Catalyst name | (V vs SCE) | (M⁻¹ s⁻¹) |
| dimethyl phthalate | −1.928 | 0.50 |
| di-isobutyl phthalate | −1.948 | 0.82 |
| dibutyl phthalate | −1.958 | 0.90 |
| phenyl benzoate | −2.026 | 2.15 |
| ethyl 3-fluorobenzoate | −2.043 | 2.43 |
| methyl 3-phenoxybenzoate | −2.085 | 3.01 |
| phenyl 4-methylbenzoate | −2.094 | 3.08 |
| methyl benzoate | −2.202 | 4.19 |
| ethyl benzoate | −2.221 | 4.21 |
| methyl 3-methylbenzoate | −2.237 | 4.44 |
| methyl 2-methylbenzoate | −2.276 | 4.74 |
| methyl 4-methylbenzoate | −2.290 | 5.04 |
| benzonitrile | −2.260 | 5.31 |
| O-tolunitrile | −2.297 | 5.60 |

The reason for selecting O-tolunitrile as catalyst is that its log k value is the highest when compared to others from Table 12. It has been assumed that the reduction of CO₂ in the presence of the catalyst happens directly at the cathode. If this is correct, then the presence of the catalyst elsewhere in the system is inconsequential. The cathode's conductive surface was limited without disrupting the overall electric field across the cell. If the field across the cell is all that is required, then the kinetics should remain unchanged. Different sizes for the surface of the cathode were tested, which was found that the smaller the surface area available for reduction, the lesser the oxalate formation.

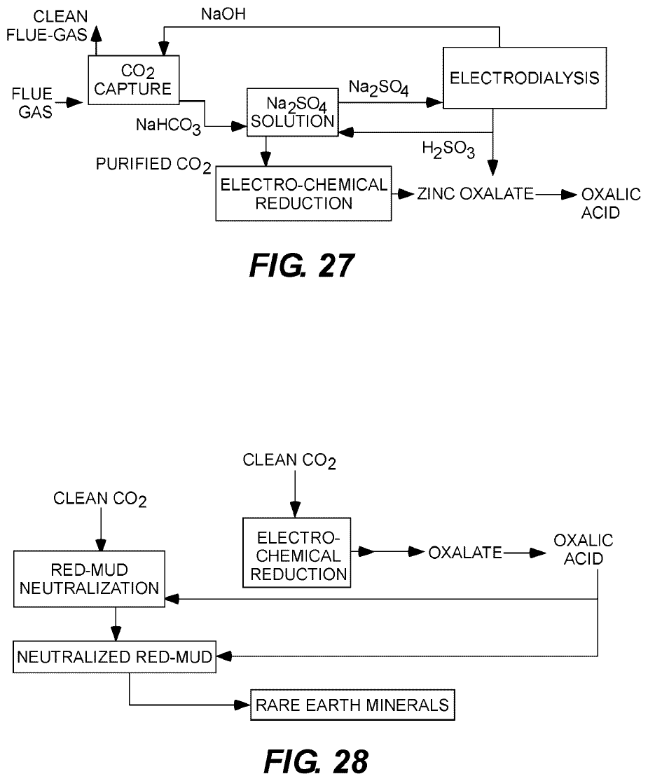

Since the aromatic nitrile catalyst is surface active, there is essentially no requirement for any internal volume beyond what is necessary to hold a few bubble-diameters of fluid and maintain conductivity between the cathode and the membrane. This would decrease the quantity of aprotic solvent required considerably, decreasing the overall cost of the process. FIG. 27 shows the further step of preparing oxalic acid from an oxalate salt precipitate.

An oxalate salt, particularly in this instance zinc oxalate, from which oxalic acid may be produced was prepared by reducing CO₂ at a Zinc/lead cathode in an organic solvent, with an addition of aromatic nitrile catalyst. Current densities of 25 mA/cm² and higher have proven effective in producing more oxalate in the solid precipitate product.

The oxalate salt generated by the electrochemical reduction can be converted to oxalic acid by the acidification of oxalate to oxalic acid with a strong acid. In some aspects, the strong acid comprises an inorganic acid. In some aspects, the strong acid comprises sulfuric acid or hydrochloric acid.

The oxalate salt generated by the electrochemical reduction can be converted to oxalic acid via electrochemical acidification. In some aspects, an electrochemical acidification unit configured to acidify the oxalate salt fed to an ion exchange region to produce the oxalic acid. For instance, an EDBM process previously described can be used to supply H⁺ and OH⁻ in situ. In this electrodialysis process, the alkali ion and the oxalate migrate to the cathode and anode, respectively, but the oxalate ions would be oxidized and decomposed by oxygen in the anodic compartment. To avoid this, cation and bipolar membranes are required. The simplest option is the use of two cation-exchange membranes as shown in FIG. 12A. Protons are provided by water splitting in the hydrogen evolution reaction on the anode. The hydroxy ions are provided by water splitting in the oxygen evolution reaction on the cathode.

IV. Utilization of Captured CO₂ and/or Oxalic Acid Formed from Captured CO₂ in Rare Earth Mineral Recovery

Captured CO₂ can be used in other applications beyond the formation of an oxalate salt or oxalic acid via the formation of an oxalate salt. For instance, captured CO₂ can be utilized in the neutralization of red mud. Red mud (RM) is the caustic waste material of bauxite ore processing for alumina extraction. During the digestion of bauxite ore in a NaOH solution at increased temperatures under pressure, red mud is a waste product after the formation of soluble sodium aluminate. The chemical composition of red mud is relatively complex, with the physical and chemical properties varying depending upon the mining areas and the production process. Red mud slurry is highly alkaline with pH values usually between 9-13 and sometimes over 13 due to the presence of NaOH and Na₂CO₃. The main constituents of red mud (% w/w) are: Fe₂O₃ (30-60%), Al₂O₃ (10-20%), SiO₂ (3-50%), Na₂O (2-10%), CaO (2-8%), and TiO₂ (trace-10%).

Red mud can be neutralized with acid neutralization, which is a very simple method based on the principle of acid-base neutralization. In some aspects of the present invention, the captured CO₂, oxalic acid, or a combination thereof, can be used to neutralize red mud.

In some preferred aspects, captured CO₂ can be mixed with red mud to provide neutralized red mud. In some preferred aspects, the captured CO₂ is mechanically mixed with the red mud to provide neutralized red mud. In some preferred aspects, the captured CO₂ is mixed with red mud to have a CO₂/red mud ratio of at least 5:1, in some aspects at least 6:1, and in some aspects at least 7:1 at a temperature of at least 45° C. and a pressure of the captured CO₂ of at least 3 MPa, in some aspects at least 3.5 MPA and in some aspects at least 4 MPA. The neutralized red mud preferably has a reduced pH, preferably a pH below 7.0, in some aspects an equilibrium pH between 6.0 and 7.0. Without wishing to be bound be theory, it is believed that the neutralization of aqueous red mud solution takes place by the following carbonation reactions of CO₂:

CO₂(aq)+OH⁻(aq)↔HCO₃⁻(aq)

HCO₃⁻(aq)↔H⁺(aq)+CO₃²⁻(aq)

NaAl(OH)₄(aq)+CO₂(aq)↔NaAl(OH)₂CO₃(s)+H₂O

3Ca(OH)₂·2Al(OH)₃(s)+3CO₂(aq)↔3CaCO₃(s)+2Al(OH)₃(s)+3H₂O

Na₆|AlSiO₄|6·2NaOH+2CO₂(aq)+Na₆|AlSiO₄|6+2NaHCO₃

The carbonic acid formed in the CO₂ bearing fluid neutralizes the bases and precipitates as sodium carbonate, calcium carbonate, magnesium carbonate and combinations thereof.

Red mud can also be neutralized with oxalic acid. In some aspects, the oxalic acid is derived from captured CO₂ using the electrochemical reduction process described above. The oxalic acid can be mixed with the red mud for the neutralization of red mud to provide neutralized red mud. In some preferred aspects, at least 10%, in some aspects at least 12%, and in some aspects at least 15% oxalic acid is combined with red mud at an elevated temperature above 75° C. for at least 30 minutes at a liquid/solid ratio of greater than 3 mL/g, in some aspects greater than 3.25 mL/g, in some aspects greater than 3.5 mL/g, in some aspects greater than 3.75 mL/g, and in some preferred aspects at least 4 mL/g. In some preferred aspects, the oxalic acid is mechanically mixed with the red mud to provide neutralized red mud.

In some preferred aspects, red mud is neutralized by the use of captured CO₂ and oxalic acid, the oxalic acid preferably derived from captured CO₂ using the electrochemical reduction process described above.

The neutralized red mud can further be treated with oxalic acid for the extraction of rare earth metals. In some preferred aspects, the oxalic acid is derived from captured CO₂ using the electrochemical reduction process described above. In some preferred aspects, the rare earth metals recovered from neutralized red mud using oxalic acid include Al, Na, Fe, Ti and rare earth elements. In some preferred aspects, the rare earth elements include lanthanum, cerium, praseodymium, neodymium, promethium, samarium, europium, gadolinium, terbium, dysprosium, holmium, erbium, thulium, ytterbium, lutetium, scandium and yttrium.

FIG. 28 is a schematic flow diagram illustrating the use of captured CO₂, such as purified CO₂ using EDBM separation as shown in more detail in FIG. 11, being used for the neutralization of red mud. FIG. 28 also illustrates that the captured CO₂ can be formed into oxalic acid via the electrochemical reduction process to form an oxalate salt. The oxalic acid can also be used for the neutralization of red mud. The oxalic acid can further be used for the extraction of rare earth minerals from the neutralized red mud.

The neutralized red mud can be used as a material, such as employed as a catalyst or catalyst support, building material including cement, permeable bricks, glass-ceramics, ceramic foam, and road base material.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. (canceled)

2. A method for the production of oxalic acid, the method comprising:
providing a feedstock of purified carbon dioxide, the feedstock of purified carbon dioxide derived from a captured carbon dioxide from an emissions source;
electrochemically reducing the feedstock of purified carbon dioxide to an oxalate salt; and
converting the oxalate salt to oxalic acid.

3. (canceled)

4. The method of claim 2, wherein the feedstock of purified carbon dioxide comprises industrial grade CO₂ having a purity of at least 99.5%, medical grade CO₂ having a purity of at least 99.5%, bone dry grade CO₂ having a purity of at least 99.8%, food grade CO₂ having a purity of at least 99.9%, beverage grade CO₂ having a purity of at least 99.9%, anaerobic grade CO₂ having a purity of at least 99.95%, or research grade CO₂ having a purity of at least 99.999%.

5. The method of claim 2, wherein the emissions source is a flue gas resulting from combustion of a fossil fuel, wood, or a renewable power source.

6. The method of claim 2, wherein the captured CO₂ is captured from a flue gas and has undergone further processing to provide a purity of at least 99.5%.

7. The method of claim 6, wherein the captured CO₂ has been captured from the flue gas using a chemical absorption capture of CO₂ using a scrubbing absorption column having a slurry scrubbing solution that is capable of capturing CO₂ from the flue gas.

8. The method of claim 7, wherein the slurry scrubbing solution comprises a sodium carbonate solution mixed with at least one frothing agent mixed, wherein the at least one frothing agent comprises at least one compound of Formula (I): 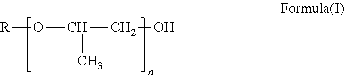
wherein R is H or CH₃, and wherein n is greater than 2 and up to 34, preferably n being between 3 and 34, more preferably n being between 3 and 8; and
wherein the at least one frothing agent present within the slurry scrubbing solution enhances the absorption rate of carbon dioxide from a gaseous feedstock by the slurry scrubbing solution to produce a resultant product compared to the absence of the at least one frothing agent within the slurry scrubbing solution.

9. The method of claim 8, wherein slurry scrubbing solution capturing CO₂ from the flue gas provides a resultant product comprising a sodium bicarbonate solution.

10. The method of claim 9, wherein the sodium bicarbonate solution is capable of being converted to a regenerated slurry scrubbing solution and the feedstock of purified carbon dioxide in a regeneration assembly, the regeneration assembly comprising a regeneration vessel.

11. The method of claim 2, wherein the captured CO₂ has been captured from the flue gas using a scrubbing absorption column containing a slurry scrubbing solution at alkaline pH conditions for the simultaneous capture of CO₂, NOₓ and SOₓ from the flue gas.

12. The method of claim 11, wherein the slurry scrubbing solution comprises a sodium carbonate solution and at least one oxidizer.

13. The method of claim 12, wherein the at least one oxidizer is chosen from the group consisting of H₂O₂, NaOCl, NaOCl₂, NaClO₃, and mixtures thereof.

14. The method of claim 12, wherein the scrubbing solution is provided at a pH in the rage of about 8 to about 13, in some aspects about 9 to about 12.5, in some aspects about 10 to about 12.2, and in some preferred aspects about 11 to about 12.

15. The method of claim 2, wherein the captured CO₂ by the slurry scrubbing solution has undergone thermal regeneration to provide the purified CO₂.

16. The method of claim 2, wherein the slurry scrubbing solution comprises a sodium hydroxide (NaOH) solution, the captured CO₂ is in the form of a metal bicarbonate solution comprising a sodium bicarbonate solution, wherein the metal bicarbonate solution is reacted with an acid reagent in a reaction tank to form the purified carbon dioxide and a resultant salt solution, the acid reagent comprising sulfuric acid, and the resultant salt solution comprising a sodium sulfate (Na₂SO₄) solution.

17. The method of claim 16, wherein the resultant salt solution is subjected to electrodialysis with bipolar membrane separation for separation of the resultant salt solution into an acid and a base, wherein the electrochemical reduction employs an electrolysis cell having a cathode and an anode, wherein the acid from electrodialysis comprises a regenerated acid reagent and wherein the base from electrodialysis comprises a regenerated slurry scrubbing solution, the regenerated acid reagent comprising sulfuric acid, and the regenerated slurry scrubbing solution comprising sodium hydroxide.

18. The method of claim 17, wherein the cathode is wrapped at least partially around the anode in a cylindrical configuration.

19. The method of claim 17, wherein the electrolysis cell is devoid of a membrane between a catholyte region and an anolyte region.

20. The method of claim 17, wherein the cathode has a metal coating on a cathode surface, that has an increased absorbing and conversion of the captured CO₂ into oxalate salt compared to the cathode without the metal coating.

21. The method of claim 17 wherein the cathode has a modified cathode surface comprising a metal coating that provides a rough surface area compared to the cathode without the metal coating, the metal coating increasing the surface area of the cathode surface compared to the cathode surface without the metal coating.

22. The method of claim 17 wherein the cathode has a modified cathode surface comprising a metal coating comprising lead, zinc, steel, silver, iron or copper.

23-30. (canceled)
